(19) United States
(12) Patent Application Publication
(10) Pub. No.: US 2003/0186941 A1
(43) Pub. Date: Oct. 2, 2003

(54) Cyclopenteneone derivatives

(76) Inventors: Stanley Michael Roberts, Parkgate (GB); Maria Gabriella Santoro, Avellino (IT); Eric Emil Anggard, Marsh Baldon (GB)

Correspondence Address: PENNIE AND EDMONDS, 1155 AVENUE OF THE AMERICAS, NEW YORK, NY, 100362711

(21) Appl. No.: 10/168,036

(22) PCT Filed: Dec. 18, 2000
(86) PCT No.: PCT/GB00/04868

(30) Foreign Application Priority Data
Dec. 16, 1999 (GB) 9929702.0

(51) Int. Cl. A61K 31/695; C07D 277/30; C07D 333/24; A61K 31/12; A61K 31/44; A61K 31/381

(52) U.S. Cl. 514/63; 514/277; 514/438; 514/679; 546/340; 548/204; 568/330; 549/79

(57) ABSTRACT

A compound having a cyclopent-2-ene-1-one ring and also having a double bond directly attached to the ring (in addition to the C═O bond of the cyclopent-1-ene-1-one ring) may be useful in treating various disorders, including viral disorders, cancers, inflammatory disorders, etc.

The present invention relates to certain cyclopentenone derivatives, their preparation and uses in medicine and other fields.

Various compounds comprising the cyclopentenone ring structure (also known as the cyclopentenone nucleus) are capable of inducing the heat shock response. The heat shock response is a finely regulated and highly conserved mechanism to protect cells against different types of injury, including extreme temperatures, oxidative stress, exposure to toxins and viral infection (1). In human cells, triggering of the heat shock response requires activation of a transregulatory protein, the heat shock transcription factor type 1 (HSF 1), which controls the expression of cytoprotective heat shock proteins (HSPs) (1). Whereas HSP induction was at first interpreted as a signal for detection of physiological stress, it is now accepted that HSPs are utilised by cells as molecular chaperones in the repair process following different types of injury to prevent damage resulting from the accumulation and aggregation of non-native proteins (1). In particular, a cytoprotective role of the heat shock protein HSP70 has now been described in a wide variety of human diseases, including ischemia, inflammation and viral infection (2-5). For these reasons HSF 1 is considered a novel, attractive target for cytoprotective and antiviral drugs. In the case of viral infection, Santoro et al. have shown that a class of prostaglandins (PGs) with potent antiviral activity function as HSP70 inducers via HSF1 activation (6,7).

The ability of prostaglandins of the A type (PGAs) to inhibit viral replication and prevent the establishment of persistent infections was first reported in 1980 (8). It is now well established that PG containing an α,β-unsaturated carbonyl group in the cyclopentane ring structure (cyclopentenone PG, cyPG) possess activity against a wide variety of DNA and RNA viruses, including herpes viruses, paramyxo viruses, orthomyxo viruses and retroviruses in in vitro and in vivo experimental models (9). The mechanism of the antiviral activity is distinct from any other known antiviral agent and involves the induction of heat shock proteins and the inhibition of the transcription factor NF-κB (nuclear factor-κB) in the infected cell.

NF-κB is an inducible eukauyotic transcription factor which has a critical role in promoting inflammation and viral replication (11). In most cells, NF-κB exists in an inactive cytoplasmic complex, whose predominant form is a heterodimer composed of p50 and p65 subunits, bound to inhibitory proteins of the IκB family, usually IκBα, and is activated in response to primary (viruses, bacteria, UV) or secondary (inflammatory cytokines) pathogenic stimuli (12). Stimulation triggers rapid phosphorylation and degradation of IκBα, resulting in NF-κB translocation to the nucleus, where the factor binds to DNA at specific κB-sites, inducing a variety of genes encoding signalling proteins. Target genes include inflammatory and chemotactic cytokines, cytokine receptors and viral genes. NF-κB is involved in many pathological events including progression of AIDS by enhancing HIV-1 transcription and is considered an attractive therapeutic target for novel antiviral and anti-inflammatory drugs (12). Santoro et al. have shown that cyclopentenone prostaglandins inhibit NF-κB activation and NF-κB dependent HIV-1 transcription in human cells, by preventing IκBα phosphorylation and degradation, and that this effect is strictly associated with HSF1 activation (11).

Santoro et al. have identified the molecular structure of natural prostaglandins responsible for HSF activation and NF-κB inhibition (13). One component of the PDA molecule, cyclopent-2-en-1-one (also known as 2-cyclopenten-1-one), at a concentration of 125-500 μM, has been shown to be able to activate HSF1 and to rapidly and selectively trigger the synthesis of cytoprotective HSP70. At the same concentration, cyclopent-2-en-1-one has been shown to be able to block NF-κB activation by chemical or physiological inducers. These effects are associated with antiviral activity during infection with rhabdoviruses (13).

Compounds in accordance with the present invention comprise a cylopent-2-ene-1-one ring and have a double bond directly attached to the ring (in addition to the C═O bond of the cyclopent-2-ene-1-one ring). The compounds also have an oxygen atom directly attached to the ring via a single bond.

According to the present invention there is provided a compound having the formula I:—

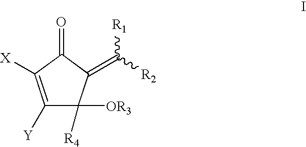

wherein:—

R₁ is H, or a substituted or unsubstituted alkyl or alkenyl group containing 1 to 3 carbon atoms;

R₂ is a substituted or unsubstituted alkyl, alkenyl, alkynyl, aryl, arallyl aralkenyl, or aralkynyl group, optionally including at least one heteroatom in its carbon skeleton, and containing 1-12 carbon atoms;

R₃ is a substituted or unsubstituted alkyl, alkenyl, alkynyl, aryl, aralkyl aralkenyl, or aralkynyl group, optionally including at least one heteroatom in its carbon skeleton, and containing 1-12 carbon atoms, or a silyl group;

R₄ is H, or an alkyl group containing 1-3 carbon atoms;

X and Y, independently, are H, a halogen, or an alkyl group containing 1-3 carbon atoms; and

R₂ can be cis- or trans- with respect to the carbonyl carbon in the cyclopentene ring.

For the avoidance of doubt, it is confirmed that the term “alkenyl” denotes an a group with one or more double bonds in its carbon skeleton and the term “allynyl” denotes a group with one or more triple bonds in its carbon skeleton. It should also be understood that, for the purposes of this specification, alkynyl groups may include both double and single bonds in their carbon skeletons.

R₂ and R₃, independently, can be substituted with one or more ═O, —OR₅, —COOR₅ and/or a halogen (preferably fluorine)group or atom, wherein R₅ is, independently, hydrogen or an alkyl group containing up to 4 carbon atoms. R₅ preferably is hydrogen or a methyl group.

R₂ and R₃, independently, can be unsubstituted.

When present, the heteroatom in the carbon skeleton of R₂ and/or R₃ is preferably oxygen, nitrogen or sulphur.

R₄ is preferably hydrogen or an unsubsituted alkyl (preferably methyl) group; hydrogen being more preferred.

R₄ is preferably hydrogen or a methyl group; hydrogen being more preferred.

Each of R₂ and R₃, independently, can be a substituted or unsubstituted, straight chain, branched and/or cyclo alkyl alkenyl, or alkynyl group.

In a preferred embodiment, R₂ is a substituted or unsubstituted heterocyclic, aralkyl or aryl group. Thus, R₂ can be a substituted or unsubstituted phenyl, thiopheneyl, or pyridinyl group. In more preferred embodiments, R₂ is an unsubstituted thiopheneyl, pyridinyl, phenyl, dimethylphenyl, halophenyl or alkoxyphenyl group. The halophenyl group is preferably a fluorophenyl group and the alkoxyphenyl group is preferably a methyloxyphenyl group.

When R₂ is a substituted or unsubstituted alkyl, alkenyl or alkynyl group it preferably includes up to 10, 9, 8, 7, 6, 5, 4, or 3 carbon atoms. In preferred embodiments, R₂ contains the 7, 3 or 2 carbon atoms and is preferably an allyl group. In especially preferred embodiments R₂ is C₇H₁₅, iso-propyl, or ethyl.

R₃ can be a substituted or unsubstituted alkyl or aralkyl group and, in preferred embodiments R₃ includes a carboxyl and/or a carbonyl group.

R₃, in preferred embodiments, is a substituted or unsubstituted straight chain, branched and/or cyclo-alkyl group. R₃ preferably contains 5, 6, 7, or 8 carbon atoms when it includes or is a cycloalkyl group and 5 or fewer carbon atoms when it is a straight chain or branched alkyl group. In other embodiments R₃ is an aralkyl group and preferably contains 6, 7, or 8 carbon atoms. When R₃ is a straight chain or branched alkyl group it, more preferably, contains 4, or 5 carbon atoms. Preferred cycloalkyl groups are cyclohexyl groups and the preferred aryl group is phenyl.

In preferred embodiments, R₃ is a succinyl (i.e., a 1-oxo-3-carboxyprop-1-yl) group, or derivative. The derivative can be a 2-methylsuccinyl (i.e., a 1-oxo-2-methyl-3-carboxyprop-1-yl) group, or a group wherein two of the carbon atoms of the succinyl moiety form part of a saturated or unsaturated ring. Thus, the derivative can be a 2-carboxyphenylcarbonyl or a 2-carboxycyclohexylcarbonyl group.

In further embodiments, R₃ includes a carbonyl group a to the oxygen atom bound to the cyclopenteneone ring.

When R₃ is a silyl group it is, preferably, a tri(organo)silyl group. Each of the organo-groups can be a substituted or unsubstituted alkyl, aryl and/or aralkyl group, optionally including at least one heteroatom in its carbon skeleton. Any combination of three such groups (e.g. one alkyl group, one aralkyl group and one aryl group; one or two alkyl groups combined with two or one aryl or arakyl groups; etc.) can be present. Where alkyl groups are present, they preferably have from 1 to 5 carbon atoms. Where aryl or alkaryl groups are present, preferably they have at least 6 or 7 carbon atoms respectively. Preferred aryl groups include phenyl groups and preferred aralkyl groups include benzyl groups. If desired, the allyl, silyl, or tri(organo)silyl groups, benzyl or phenyl groups can include various hetero atoms and/or groups (e.g. one or more hydroxyl groups and/or halogen atoms may be present in them). Thus the organo-groups can be substituted with one or more ═O, —OR₅, COOR₅ and/or halogen (preferably fluorine) group or atom, wherein R₅ is, independently, hydrogen or an alkyl group containing up to 4 carbon atoms. R₅ preferably is hydrogen or a methyl group.

Compounds in accordance with the invention exist in the form of a least two enantiomers and all such enantiomers, unequal mixtures thereof and racemates are encompassed by the present invention. Both R- and S-enantiomers of the compounds of the present invention are useful. They can each be provided in a form substantially free of the other enantiomer (e.g. at least 75%, 85%, 90%, 95% or 99% free (w/w)). Mixtures of enantiomers (e.g. racemic mixtures) may however also be used.

Many compounds in accordance with the invention exist in both E and Z forms, i.e. with R₂ being cis- or trans to the carbonyl carbon in the cyclopenteneone ring. The present invention encompasses all such individual isomers and mixtures thereof.

Compounds of the present invention have significant differences from the punaglandins and prostaglandins that have been disclosed previously for therapeutic purposes. In particular, it can be noted that compounds of the present invention do not require the presence of two long aliphatic lateral side chains (usually each comprising more than 7 carbon atoms) attached to the cyclopentenone ring structure. Thus, although two such chains can be included if desired, preferred compounds of the present invention do not include the presence of the two long aliphatic lateral side chains associated with prostaglandins.

The preferred embodiments of R₂ and R₃ are those illustrated in the following formulae, especially in formulae CTC-31, 2, 3, 4, 5, 6 and 45. Each of the embodiments of R₂, given below, can be used with alternative embodiments of R₃ to those illustrated in the individual formulae and vice versa.

Preferred compounds in accordance with the invention include the following:—

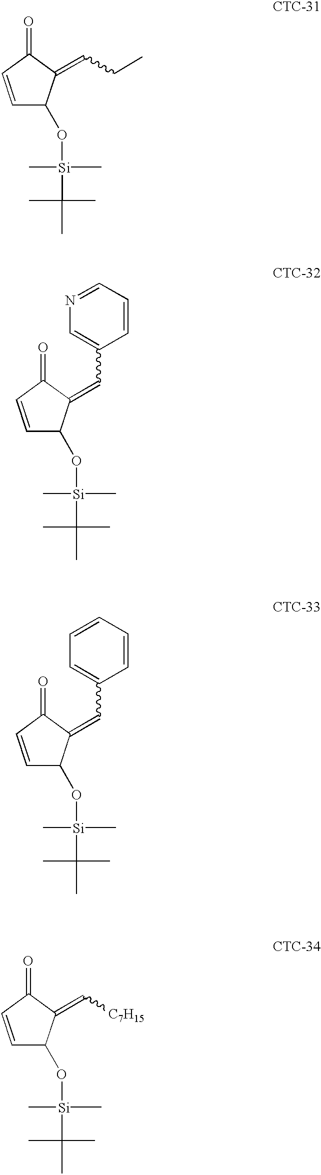

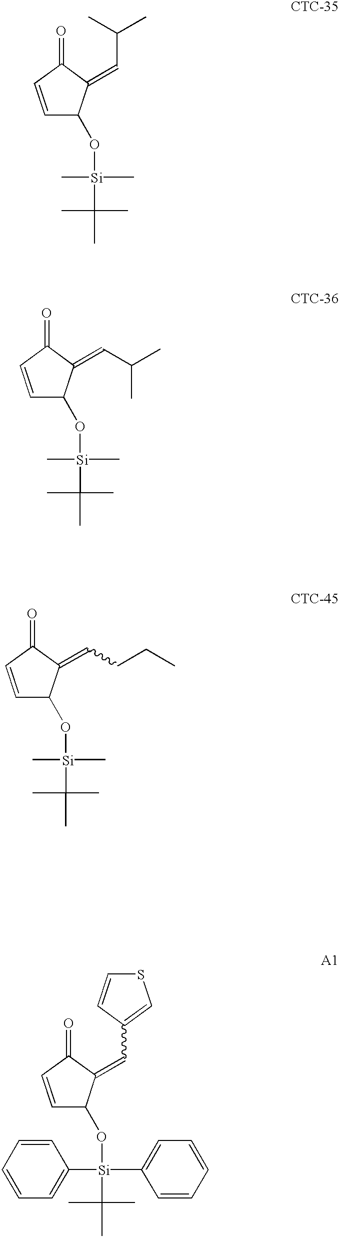

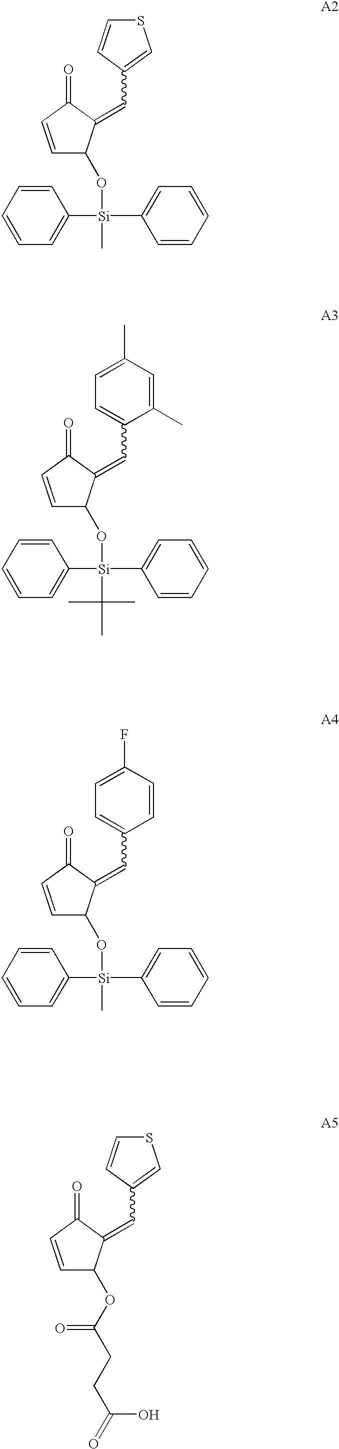

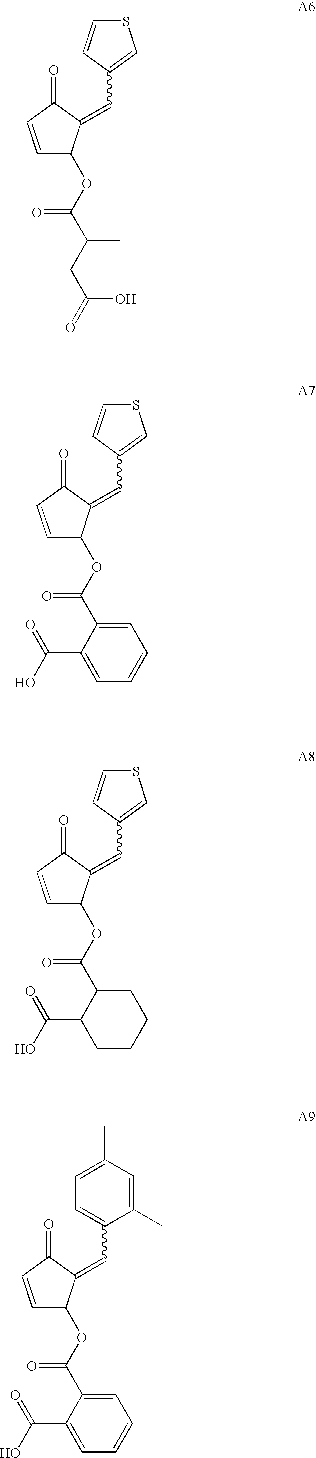

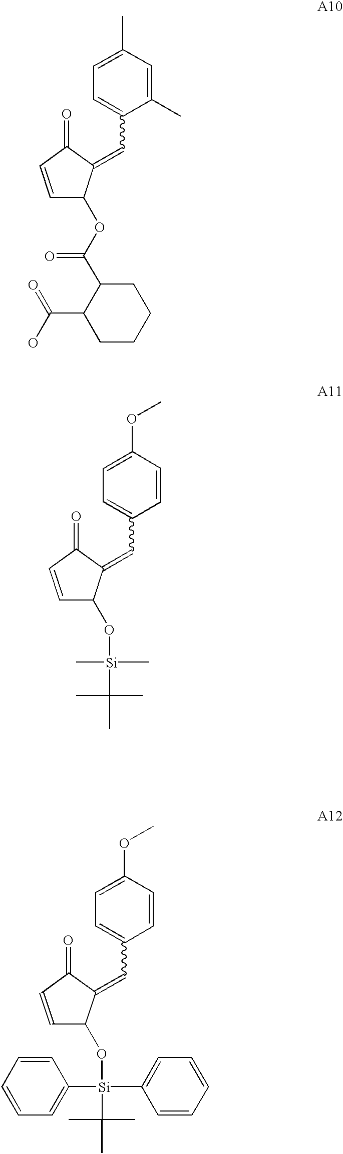

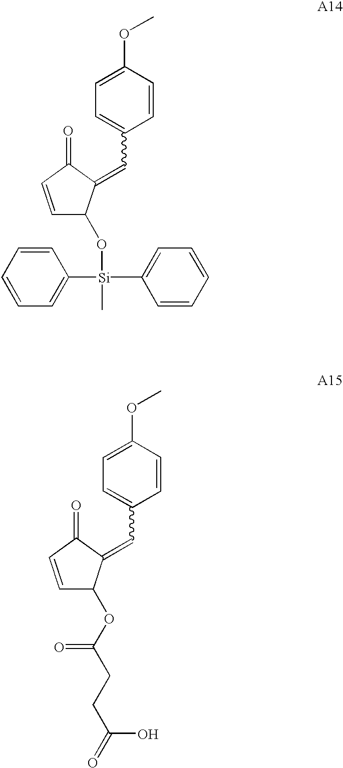

In the foregoing formulae and elsewhere in this specification, groups joined by bonds shown as [symbol] can be orientated in either the cis- or trans-configurations and both such forms (i.e., the E and Z forms) of the depicted compounds lie within the scope of the present invention.

In a second aspect, the present invention provides a method for preparing compounds in accordance with the first aspect of the invention. Such methods comprise reacting a compound of formula II:—

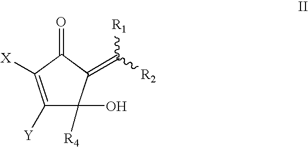

with a silyl chloride, succinic anhydride, or a derivative or succinic anhydride, preferably in the presence of a base and, more preferably, also in the presence of an alkyl amino pyridine. The alkyl amino pyridine is preferably dimethyl amino pyridine.

In formula II, R₁ and R₂ are as defined above and the silyl group in the silyl chloride is also as defined above. The succinic anhydride derivative is selected to provide the required group R₃.

Compounds of formula II can be prepared by the method described in example 7 below, or by using general method (b), which is described in example 1 below, in which Q is either a hydroxyl group, or is reduced to a hydroxyl group before commencing.

In accordance with a third aspect of the present invention, there is provided a method of preparing compounds of formula III:—

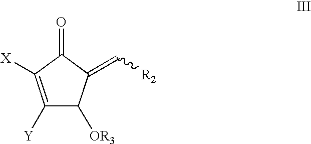

including the steps of:—

(a) reacting a compound of formula IV

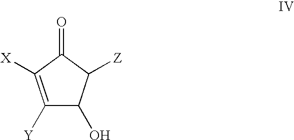

with a silyl chloride, succinic anhydride or a succinic anhydride derivative, preferably in the presence of a base and, more preferably, also in the presence of an alkyl amino pyridine (the alkyl amino pyridine is preferably dimethyl amino pyridine), to prepare a compound of formula V:—

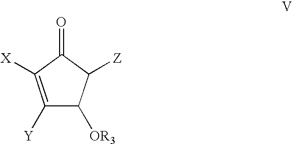

and

(b) reacting a compound of formula VI:—

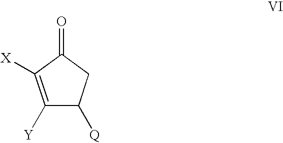

with R₂ CHO in the presence of a base to give a compound of formula VII:—

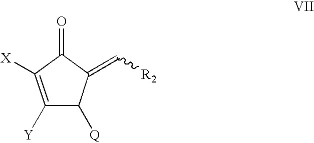

wherein;

when step (a) is carried out before step (b) Z is hydrogen, Q is OR₃ and the bonds between Z, Q and the cyclopentene ring are single bonds;

when step (b) is carried out before step (a), Q is an oxygen atom or a hydroxyl group, Z is CR₂ and the carbon atom in the CR₂ group is bonded to the cyclopentene ring by a double bond;

when Q is oxygen, the bond between the oxygen atom and the cyclopentene ring is a double bond and it is reduced to a hydroxyl group before step (a) is carried out;

X, Y, R₂, R₃ and the silyl group in the silyl chloride are as defined above; and the succinic anhydride derivative is selected to provide the required group R₃.

In further aspects, the present invention provides compounds in accordance with the first aspect of the invention for use in medicine, particularly for the therapeutic treatment of the human or animal body, or for use in a diagnostic method practised on the human or animal body. Therapeutic and diagnostic methods, involving the use of compounds in accordance with the present invention, are also within the remit of the invention.

The use of compounds in accordance with the first aspect of the invention for the manufacture of medicaments for use in therapeutic or diagnostic methods to be practised on the human or animal body lie within the scope of a further aspect of the invention.

Preferred therapeutic and diagnostic applications for compounds in accordance with the invention are discussed in the following section.

A compound of the present invention will preferably have activity in respect of one or more of the following:

(a) activating HSF

(b) inhibiting NF-κB

(c) inhibiting the replication of HSV-1

(d) inhibiting the replication of Sendai virus

(e) inhibiting the influenza virus.

A skilled person can readily assay for the above activities. Examples of suitable assay procedures are set out herein in Examples 9, 10, 11, and 12.

Compounds that have greater activity than cyclopent-2-en-1-one (at least at certain concentrations) are most preferred. Such compounds may for example have a level of activity that is at least twice the level of cyclopent-2-en-1-one. More preferably, it is at least ten times that of cyclopent-2-en-1-one.

In a further aspect of the present invention, compounds in accordance with the first aspect of the invention can be used to treat plant diseases, particularly viral infections.

Medical Uses

Compounds of the present invention may be used for any desired therapeutic purpose. Preferred treatments are human treatments, although veterinary treatments are also within the scope of the present invention. The treatment may be prophylactic or may be in respect of an existing condition.

Treatments are desirably of disorders which can be treated in a host by the activation of a heat shock transcription factor (e.g. HSF1), by the induction of heat shock proteins (e.g. hsp70) and/or by the inhibition of NF-κB.

Various preferred treatments are discussed below. (It should be appreciated that certain disorders—e.g. cancers—may be mediated by viruses and by non-viral factors. In the absence of any indication to the contrary, treatment of any given disorder is covered whether or not the disorder is mediated by viruses. It should also be appreciated that there is some overlap between the various categories of treatment discussed—i.e. the categories are not intended to be mutually exclusive).

1. Treatment of Viral-Mediated Disorders

NF-κB is implicated in the pathogenesis of certain viral infections. It is known that heat shock proteins (e.g. HSP70) can offer protection against the pathogenesis of viral infection. Compounds of the present invention may be active in reducing the replication of viruses.

Compounds of the present invention may be useful in treating viral-mediated disorders. These include disorders mediated by RNA viruses, as well as disorders mediated by DNA viruses.

Examples of viral disorders that may be treated using compounds of the present invention include disorders mediated by: retroviruses (e.g. HIV1), herpes viruses (e.g. HSV-1, CMV, HHV8, HSV-2), paramyxo and orthomyxo viruses (as illustrated by Sendai viruses and including influenza viruses), rhabdoviruses (e.g. vesicular stomatitis virus, rabies viruses), picornaviruses (e.g. rhinoviruses, hepatitis A and polio viruses), hepadnaviruses (e.g. hepatitits B viruses), togaviruses (e.g. rubella viruses), poxviruses (e.g. molluscum contagiosum virus).

Additional viral disorders that may be treated using compounds of the present invention include: filoviruses (e.g. Ebola virus), bunyaviruses (e.g. hantaviruses), arenaviruses (e.g. lassa fever virus), flaviviruses (e.g. yellow fever and hepatitis C viruses).

Compounds of the present invention may be particularly useful in treating viral and other disorders affecting aquatic organisms (e.g. fish, crustaceans, etc.). Such disorders include disorders mediated by the snout ulcer virus, by the iridovirus, by the lymphocystis disease virus, etc.

Compounds of the present invention may therefore be used in aquaculture. They may be used in food for aquatic organisms. Such food is within the scope of the present invention. It will generally be sold in sealed containers and labelled appropriately (e.g. as fish food, food for crustaceans, food for aquatic organisms, etc.). Alternatively, compounds of the present invention may be used for water treatment or for direct application to aquatic organisms. Such compounds do not therefore need to be present in foodstuffs in order to be useful in aquaculture.

2. Treatment of Bacterial-Mediated Disorders

NF-κB is activated in response to bacterial infections.

Compounds of the present invention can be useful in treating disorders arising from such infections—e.g. in treating NF-κB stimulated inflammation. Most commonly this will arise due to infection with gram negative bacteria. However it may also arise due to infection with gram positive bacteria (e.g. S. aureus).

3. Treatment of Disorders Mediated by Radiation

NF-κB is activated in response to radiation (e.g. UV-radiation).

Compounds of the present invention can be useful in treating disorders mediated by radiation. Such disorders include cell and tissue trauma, cell and tissue ageing and cancer (e.g. skin cancer).

4. Treatment of Inflammation and of Disorders of the Immune System

NF-κB is activated in response to inflammatory cytokines. It is believed to be an early mediator of the immune and inflammatory responses.

Compounds of the present invention can be useful in treating immune disorders (e.g. auto-immune disorders) and in treating inflammatory disorders.

Examples of specific inflammatory disorders and disorders of the immune system that may be treated with compounds of the present invention include rheumatoid arthritis, multiple sclerosis, adult respiratory distress syndrome, hepatitis and/or cirrhosis, vascular inflammation (including lupus erythematosis disseminata), and inflammatory disorders of the gastro-intestinal tract (e.g. ulcers).

5. Treatment of Ishemia and Arteriosclerosis

NF-κB is implicated in cell proliferation.

Compounds of the present invention can be useful as anti-proliferatives. They are therefore useful in treating, inflammatory granulomas, neointimal proliferation in arterial and venous restenosis, and cancers (including lymphomas, leukemias, sarcomas, carcinomas and melanomas).

6. Treatment of Disorders Involving Damage to or Killing of Cells

Heat shock proteins are known to provide a cytoprotective effect.

Compounds of the present invention can be useful in treating disorders involving damage to or killing of cells.

These disorders include chemical toxicity (e.g. due to ingestion of toxins, such as paraquat, or to overdosing with medicaments, such as paracetamol), oxidative cell damage, cell and tissue ageing trauma, hepatitis diabetes and the effect of burns. The inventive compounds, also, can be used to combat the effects of ageing in a human or animal, and to promote wound healing.

Other conditions of this general nature, that can be treated using compounds of the present invention, include oxidative stress and degenerative diseases, especially neuro-degenerative diseases such as BSE, new variant CJD and Alzheimer's disease.

7.Other Treatments

Cyclopentenone prostaglandins are of known utility in stimulating peroxisome proliferator activated receptors (PPARs). Compounds of the present invention, thus, can be useful in treating diabetes (including complications arising therefrom). Compounds of the present invention can also be used in the treatment of disorders in which calcium loss or deficiency is implicated or involved (including bone disorders, skeletal disorders, dental disorders, developmental disorders, etc.).

Routes of Administration

A medicament will usually be supplied as part of a pharmaceutical composition, which may include a pharmaceutically acceptable carrier. This pharmaceutical composition will generally be provided in a sterile form. It may be provided in unit dosage form. It will generally be provided in a sealed container, and can be provided as part of a kit. Such a kit is within the scope of the present invention. It would normally (although not necessarily) include instructions for use. A plurality of unit dosage forms may be provided.

Pharmaceutical compositions within the scope of the present invention may include one or more of the following: preserving agents, solubilising agents, stabilising agents, wetting agents, emulsifiers, sweeteners, colourants, odourants, salts (compounds of the present invention may themselves be provided in the form of a pharmaceutically acceptable salt—as explained in greater detail below), buffers, coating agents or antioxidants. They may also contain other therapeutically active agents in addition to a compound of the present invention.

Compounds of the present invention may themselves be provided in any suitable form—i.e. they may be used as such or may be used in the form of a pharmaceutically effective derivative. For example they may be used in the form of a pharmaceutically acceptable salt or hydrate. Pharmaceutically acceptable salts include alkali metal salts (e.g. sodium or potassium salts), alkaline earth metal salts (e.g. calcium or magnesium salts) aluminium salts, zinc salts, ammonium salts (e.g. tetra-alkyl ammonium salts), etc. Inorganic acid addition salts (e.g. hydrochlorides, sulphates, or phosphates) or organic acid addition salts (e.g. citrates, maleates, fumarates, succinates, lactates, propionates or tartrates) may be used.

Pharmaceutical compositions of the present invention may be provided in controlled release form. This can be achieved by providing a pharmaceutically active agent in association with a substance that degrades under physiological conditions in a predetermined manner. Degradation may be enzymatic or may be pH-dependent.

Pharmaceutical compositions may be designed to pass across the blood brain barrier (BBB). For example, a carrier such as a fatty acid, inositol or cholestrol may be selected that is able to penetrate the BBB. The carrier may be a substance that enters the brain through a specific transport system in brain endothelial cells, such as insulin-like growth factor I or II. The carrier may be coupled to the active agent or may contain/be in admixture with the active agent. Liposomes can be used to cross the BBB. WO91/04014 describes a liposome delivery system in which an active agent can be encapsulated/embedded and in which molecules that are normally transported across the BBB (e.g. insulin or insulin-like growth factor I or II) are present on the liposome outer surface. Liposome delivery systems are also discussed in U.S. Pat. No. 4,704,355.

A pharmaceutical composition within the scope of the present invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) routes. Such a composition may be prepared by any method known in the art of pharmacy, for example by admixing one or more active ingredients with a suitable carrier.

Different drug delivery systems can be used to administer pharmaceutical compositions of the present invention, depending upon the desired route of administration. Drug delivery systems are described, for example, by Langer (Science 249, 1527-1533 (1991)) and Illum and Davis (Current Opinions in Biotechnology 2m 254-259 (1991)). Different routes of administration for drug delivery will now be considered in greater detail.

(i) Oral Administration

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, maize starch or derivatives thereof, stearic acid or salts thereof. Soft gelatine capsules may comprise vegetable oils, waxes, fats, semi-solid, or liquid polyols etc. Solutions and syrups may comprise water, polyols and sugars. For the preparation of suspensions, oils (e.g. vegetable oils) may be used to provide oil-in-water or water-in-oil suspensions.

An active agent intended for oral administration may be coated with or admixed with a material that delays integration and/or absorption of the active agent in the gastrointestinal tract (e.g. glyceryl monostearate or glyceryl distearate may be used).

Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

(ii) Transdermal Administration

Pharmaceutical compositions adapted for transdermal administration may be provided as discrete patches intended to remain in intimate contact with the epidermis of the recipient for a prolonged period of time. For example, the active ingredient may be delivered from the patch by iontophoresis. (Iontophoresis is described in Pharmaceutical Research, 3(6):318 (1986).

(iii) Topical Administration

Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. Here the active ingredient can be dissolved or suspended in a suitable carrier, e.g. in an aqueous solvent. Pharmaceutical compositions adapted for topical administration in the mouth include lozenges, pastilles and mouthwashes.

(iv) Rectal Administration

Pharmaceutical compositions adapted for rectal administration may be provided as suppositories or enemas.

(v) Nasal Administration

This includes not only administration to the nasal cavity, but also administration via the nasal cavity to another location—e.g. to the lungs.

Pharmaceutical compositions adapted for nasal administration may use solid carriers—e.g. powders (preferably having a particle size in the range of 20 to 500 microns). Powders can be administered in the manner in which snuff is taken, i.e. by rapid inhalation through the nose from a container of powder held close to the nose. Compositions adopted for nasal administration may alternatively use liquid carriers—e.g. include nasal sprays or nasal drops. These may comprise aqueous or oil solutions of the active ingredient.

Compositions for administration by inhalation may be supplied in specially adapted devices—e.g. in pressurised aerosols, nebulizers or insufflators. These devices can be constructed so as to provide predetermined dosages of the active ingredient.

(vi) Vaginal Administration

Pharmaceutical compositions adapted for vaginal administration may be provided as pessaries, tampons, creams, gels, pastes, foams or spray formulations.

(vii) Parenteral Administration

Pharmaceutical compositions adapted for parenteral administration include aqueous and non-aqueous sterile injectable solutions or suspensions. These may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water for injections, immediately prior to use. Extemporaneous injection solutions and suspensions may be prepared from sterile powders, granules and tablets.

From the above description it will be appreciated that compositions of the present invention can be formulated in many different way. However, preferred compositions of the present invention are in the form of topical formulations.

Dosages

Dosages of a compound of the present invention can vary between wide limits, depending upon the nature of the treatment, the age and condition of the individual to be treated, etc. and physician will ultimately determine appropriate dosages to be used.

However, without being bound by any particular dosages, a daily dosage of a compound of the present invention of from 110 g to 100 mg/kg body weight may be suitable.

More preferably the dosage is from 5 to 50 mg/kg body weight/day. The dosage may be repeated as often as appropriate. If side effects develop, the amount and/or frequency of the dosage can be reduced, in accordance with good clinical practice.

Research Uses

Compounds of the present invention are useful in research. For example, they can be used as research tools for the analysis of one or more of the following: HSF, NF-κB, the heat shock response, viral replication, viral-mediated disorders, bacterial-mediated disorders, disorders mediated by radiation (e.g. by UV-radiation), inflammatory disorders, disorders of the immune system, ischemia, arteriosclerosis, disorders involving cell proliferation (e.g. cancers), disorders involving damage to, or killing of cells (e.g. oxidative cell damage), and diabetes.

Other Uses

Compounds of the present invention can also be useful in treating plant viral disorders. Given that the basic mechanism of the heat shock response are believed to operate in a similar fashion in plants and animals and that it is reasonable to expect that direct antiviral effects will be produced by the compounds of invention in a similar fashion in plants and animals, the use of compounds of the present invention in treating viral infections of plants is within the scope of the present invention. These infections include, but are not limited to, infections by plants of geminiviruses, rhabdoviruses, caulimoviruses, bromoviruses, tobramoviruses, potyviruses and potexviruses. The use of compounds of the present invention in treating infections by viroids (including, but not limited to, potato spindle tumour viroid, hop stunt viroid, and coconut cadang viroid) is also within the scope of the invention.

### EXAMPLES

### Example 1

Synthesis of Substituted Cyclopentenone Analogues

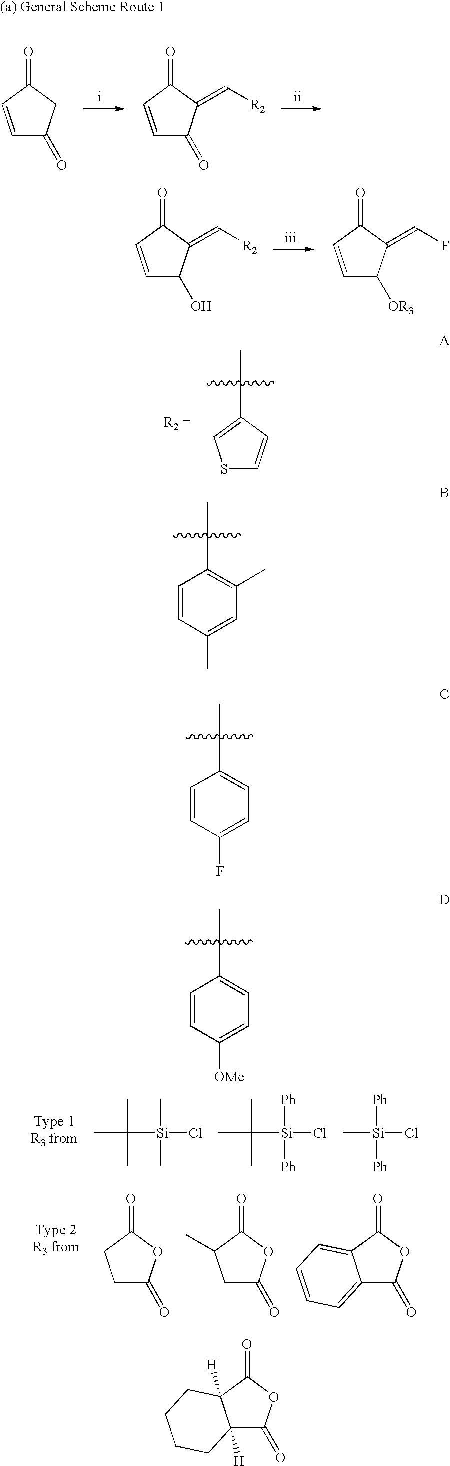

i) R₂CHO (5 eq), BF₃OEt₂ (20 eq), Et₂O, reflux, 2 hrs. ii) CeCl₃7H₂O (1 eq), NaBH₄ (0.5 eq), THF/MeOH, −40° C., 30 mins. iii) R₃SiCl or succinic anhydrides (1 eq), DIEA, DMAP, DMF, RT, 18 hours (over night).

Step (i);

Preparation of 5-Arylidene-2-cyclopentene-1,4-diones (A-D)

Commercial 2-cyclopentene-1,4-dione was first purified by dissolving in dry dichloromethane and filtering off the polymeric by-product. The filtrate was evaporated to give pure compound as a yellow crystalline solid. Boron trifluoride diethyl ether complex (20 eq) was added to a solution of the diketone (1 g) and aldehyde (5 eq) in anhydrous diethyl ether (25 ml). The mixture was heated under reflux for between 1-2 hours depending on the nature of R₁. The reaction was monitored by TLC. Upon completion the reaction was left to cool for 1 hour and diethyl ether (25 ml) was added. The mixture was poured into chilled saturated sodium chloride solution (30 ml) and ethyl acetate was added to dissolve any solids in the organic layer. The organic layer was separated and washed with saturated sodium chloride (2×25 ml), dried over magnesium sulfate and the solvent removed under vacuum. The residue was washed with successive portions of iso-hexane to remove excess aldehyde. The residual solvent was removed by high vacuum to give pure product.

A ¹H NMR (250 MHz., CDCl₃) δ 7.21-7.3 (3H, m); 7.76 (1H, s); 7.82 (1H, dd); 8.00 (1H, d).

B ¹H NMR (250 MHz., CDCl₃) δ 2.39 (3H, s); 2.5 (3H, s); 7.10-7.20 (2H, m); 7.26-7.3 (2H, m); 7.95 (1H, s); 8.4 (1H, d).

C ¹H NMR (250 MHz., CDCl₃) δ 7.2 (2H, dd); 7.3 (2H, dd); 7.6 (1H, s); 8.4 (2H, dd).

D ¹H NMR (25-MHz., CDCl₃) δ 3.92 (3H, s); 7.0 (2H, dd); 7.3 (2H, dd); 7.6 (1H, s); 8.4 (2H, dd).

Step (ii);

Preparation of 5-Arylidene-2-cyclopentenone-4-ol (A-D)

A solution of cerium chloride heptahydrate (1 eq) in methanol (concentration 0.5 mol 1⁻¹) was added to a solution of 5-arylidene-2-cyclopentenone-1,4-dione (1 g) in THF (concentration 2 mol 1⁻¹). The reaction was cooled to −40° C. with a water/acetonitrile bath. NaBH₄ (0.5 eq) was added portionwise over 10 minutes and the reaction was stirred for a further 20 minutes. Upon completion of reaction diethyl ether (50 ml) was added and the reaction was stirred as −40° C. for a further 10 minutes. The reaction was cold filtered through a plug of silica and the solvent removed under vacuum. The crude product was dissolved in methanol and pre-absorbed into silica. The compound was purified by flash column chromatography (1:1 ethyl acetate:hexane) to give pure product.

A ¹H NMR (250 MHz., CDCl,) δ 5.23 (1H, s); 6.45 (1H, d); 7.12 (1H, dd); 7.28 (1H, s); 7.42 (1H, dd); 7.57 (1H, d); 7.68 (1H, d).

B ¹H NMR (250 MHz., CDCl₃) δ 2.40 (6H, s); 5.28 (1H, d); 6.43 (1H, d); 7.00 (2H, d); 7.27 (1H, d); 7.43 (1H, dd); 7.71 (1H, d).

D ¹H NMR (250 MHz., CDCl₃) δ 3.86 (3H, s_{—}; 5.22 (1H, d); 6.42 (1H, d); 6.89 (2H, d); 7.02 (1H, s); 7.39 (1H, dd); 8.17 (2H, d).

NOTE alcohol C could not be purified from starting material and was used crude in the final step.

Step (iii);

Preparation of Target Molecules Using Type 1 Examples of R₃

The alcohol (50 mg) was dissolved in anhydrous DMF (1 ml). The solution was cooled to 0° C. and DIEA (2 eq), DMAP (0.05 eq) and silyl chloride (1 eq) were added. The reaction was allowed to warm up to ambient temperature overnight and 1 ml of distilled water and 1.5 ml of diethyl ether were added. The reaction was shaken and the organic layer separated, washed with water (2 Ml), saturated ammonium chloride (2 ml) and brine (2 ml). The organic extracts were dried over magnesium sulfate and the solvent removed under vacuum. Reactions were purified using preparative HPLC.

Compounds synthesised using this route include compounds A1, 2, 3, 4, 11, 12, and 14 referred to above.

Step (iv);

Preparation of Target Molecules Using Type 2 Examples of R₃

The alcohol (50 mg) was dissolved in anhydrous DMF (1 ml). DIEA (1.5 eq), DMAP (0.05 eq) and succinic anhyride (1 eq) were added. The reaction was stirred at room temperature overnight. Water (1 ml) and 2M HCl (0.5 ml) were added and the reaction was shaken. The aqueous layer was extracted with 2 ml of ethyl acetate. The organic extracts were dried over magnesium sulphate and the solvent removed under vacuum. Reactions were purified using preparative HPLC.

Compounds synthesised using this route include compounds A5, 6, 7, 8, 9, 10 and referred to above.

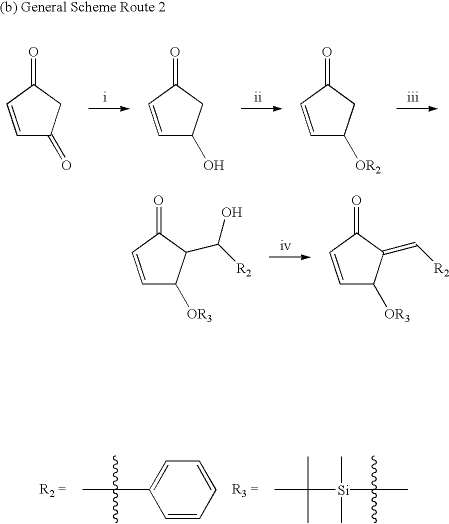

i) CeCl₃.7H₂O (1 eq), NaBH₄ (0.5 eq), MeOH, 40° C., 30 mins. ii) TBDMSCl (1 eq), DIEA (2 eq), DMAP (0.05 eq), DMF, 0° C-RT, ON. iii) LDA (1.1 eq), Benzaldehyde (1 eq), THF, −78° C., 1 hr. iv) PTSA (0.05 eq), toluene, reflux, 15 hrs.

Step (i);

Preparation of 4-hydroxy-2-cyclopentene-1-one

A solution of cerium heptahydrate (7.76 g) in methanol (30 ml) was added to a solution of 2-cyclopentene-1,4-dione (2 g) in methanol (10 ml). The reaction was cooled to 0° C. NaBH₄ (400 mg) was added portionwise over 20 minutes and the reaction was stirred for a further 1.5 hrs. Upon completion of reaction diethyl ether (50 ml) was added and the reaction was stirred at 0° C. for a further 10 minutes. The reaction was cold filtered through a plug of silica and evaporated in vacuo. The crude product was dissolved in methanol and pre-absorbed onto silica. The compound was purified by flash column chromatography (1:1 ethyl acetate:hexane) to give 620 mg product (30%).

¹H NMR (250 MHz., CDCl₃) δ 2.33 (1H, dd); 2.80 (1H, dd); 5.0 (1H, m); 6.23 (1H, dd); 7.6 (1H, dd).

Step (ii);

Preparation of 4-[(tert-butyldimethylsilyl)oxy]-2-cyclopentene-1-one

The alcohol (620 mg) was dissolved in anhydrous DMF (6 ml) and stirred under argon at 0° C. DIEA (2 eq), TBDMSCl (1 eq) and DMAP (0.05 eq) were added and the reaction was allowed to stir overnight, slowly warming to ambient temperature. The mixture was diluted with diethyl ether (50 ml), washed with water (50 ml) and brine (50 ml), then dried over magnesium sulfate. The solvent was evaporated in vacuo to give a red oil (1 g). The crude product was purified by vacuum distillation at 100° C., 0.06 mm Hg to give the product as a pale yellow oil (705 mg, 52%o).

¹H NMR (250 MHz., CDCl₃) δ 0.00 (6H, d); 0.65 (9H, s); 2.1 (1H, dd); 2.6 (1H, dd); 4.84 (1H, m); 6.04 (1H, dd); 7.35 (1H, dd).

Steps (iii) and (iv);

Preparation of 5-benzylidene-4-[(tert-butyldimethylsilyl)oxy]-2-cyclopentene-1-one (CTC-33)

A 2.3M solution of lithium diisopropylamine (450 ul) was cooled under argon to −78° C. To this was added a solution of 4-[(tert-butyldimnethylsilyl)oxy]2-cyclopentene 1-one (200 mg) in anhydrous THF (1 ml). The reaction was stirred at −78° C. for 10 minutes to from the enolate. A solution of benzaldehyde (100 mg) in THF (1 ml) was added dropwise. The cooling bath was removed and the reaction mixture allowed to warm over 40 minutes. The reaction was quenched by addition of saturated sodium hydrogen carbonate (5 ml) and extracted with diethyl ether (5 ml). The organic extracts were dried with magnesium sulfate and evaporated in vacuo to give crude product. This was purified by flash column chromatography (2:8 ethyl acetate/hexane) to give the hydrated product (52 mg, 17% yield).

¹H NMR (250 MHz., CDCl₃) δ-0.3 (3H, s); 0.00 (3H, s); 0.79 (9H, s); 1.6 (1H, s); 2.43 (1H, d); 2.77 (1H, t); 5.0 (1H, m); 5.47 (1H, m); 6.26 (1H, d); 7.3-7.5 (5H, m).

The hydrated product was dissolved in toluene (20 ml) and PTSA (0.05 eq) was added. The mixture was heated to reflux overnight. The solution was allowed to cool, washed with saturated sodium hydrogen carbonate (2 ml), brine (2 ml) and dried over magnesium sulfate. The solvent was evaporated in vacuo and a clear oil was obtained which crystallised on standing to give pure product (25 mg, 51%).

¹H NMR (250 MHz., CDCl)o 5.83 (1H, m); 6.6 (1H, dd); 7.5 (4H, m); 7.63 (1H, m); 7.89 (2H, m).

### Example 2

Synthesis of 4-tert.butyldimethylsilyloxy-5-(prop-1-ene)-cyclopent-2-ene-1-one (CTC-31)

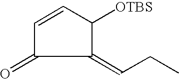

The starting material, 4-tert.butyldimethylsilyloxy-cyclopent-2-ene-1-one, was prepared as described above and according the literature procedure known to persons skilled in the art. In a 100 ml 3-neck round bottom flask, under nitrogen atmosphere at −78° C., 5 ml tetrahydrofuran CMF) was added followed by addition of 15 ml lithium diisopropylamide*. To the reaction mixture, 6.36 gm of 4-tert.butyldimethylsilyloxy-cyclopent-2-ene-1-one in 10 ml. THF was slowly added and stirred for 30 min maintaining −78° C. 6.96 gm of propionaldehyde was rapidly added and stirred for 3 hrs. Reaction mixture was quenched slowly under stirring with pre-cooled aqueous solution of saturated ammonium chloride. It was extracted with ether (3×100 ml). The combined organic layer was washed with cold 0.1 N HCl (2×100 ml) and brine (2×100 ml). Organic layer was dried over anhydrous sodium sulfate and concentrated under reduced pressure. After filtering through silica gel, the compound was taken in pyridine (5 ml) and acetic anhydride (1.2 gm) was added to it. 4-dimethyl amino pyridine (60 mg) in pyridine (5 ml) was added to the reaction mixture. The reaction mixture was heated to 60° C. for 3 hrs and cooled to room temperature. Reaction mixture was poured in cold water (100 ml.) and extracted with ether (2×100 ml.). Organic layer was separated and washed with 0.1 N HCl (2×75 ml.) and brine (2×100 ml.). The organic layer was dried over anhydrous sodium sulfate and concentrated under reduced pressure. Purification was done by column chromatography to give 67 mg. of oily title compound. * Lithium diisopropylamide is 2.0 M solution in heptane/tetrahydrofuran/ethylbenzene from Aldrich Chemical Catalog No. 36,179-8.

IR (KBr, Cm⁻¹): 2957, 2930, 2887, 2857, 1712 and 1666.

¹H NMR (CDCl₃, 400 MHz) δ: 7.38 (2H, m), 6.65 (1H, t, J=7.72 Hz), 6.40 (1H, d, J=5.92 Hz), 5.38 (1H, s), 2.0-2.5 (2H, m), 1.13 (3H, t, J=7.52), 0.94 (9H, s), 0.19 (3H, s), 0.18 (3H, s)

Mass (M/z): 253 (M⁺+1), 121 (M⁺−-OTBS)

Wherever used in this specification, the term “OTBS” denotes a tert.butyldimethylsilyloxy group.

### Example 3

Synthesis of 4-tert.butyldimethylsilyloxy-5-[methylene-(2-pyridyl)]-cyclopent-2-ene-1-one (CTC-32)

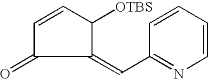

This compound was produced using the method described in Example 2 above, but with pyridine-2-aldehyde substituted for the propionaldehyde used in the latter.

IR (KBr, Cm-1): 2954, 2929, 2887, 2857, 1701 and 1642

¹H NMR (CDCl₃, 400 MHz) δ: 8.73 (1H, d, J=4.38 Hz), 7.75 (1H, t, J=7.7 Hz), 7.67 (1H, d, J=7.7 Hz), 7.60 (1H, d, J=5.98 Hz), 7.43 (1H, s), 7.26 (1H, m), 6.5 (1H, d, J=5.88 Hz), 6.07 (1H, s), 0.78 (9H, s), 0.08 (3H, s), 0.01 (3H, s)

Mass (M/z): 302 (M⁺+1)

### Example 4

Synthesis of 4-tert.butyldimethylsilyloxy-5-[methyl ene-(phenyl)]-cyclopent-2-ene-1-one

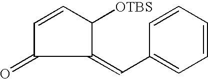

This compound was produced using the method described in Example 2 above, but with benzaldehyde substituted for the propionaldehyde used in the latter.

IR (KBr, Cm⁻¹): 2953, 2931, 2854, 2683, 1695 and 1635.

¹H NMR (CDCl₃, 400 MHz) δ: 7.82 (2H, d, J=1.76 Hz), 7.60 (1H, d, J=7.76 Hz), 7.35-7.48 (4H, m), 6.52 (1H, d, J=6.0 Hz), 5.75 (1H, s), 0.81 (9H, s), 0.09 (3H, s), 0.08(3H, s).

Mass (M/z): 301 (M+1), 169 (M⁺−-OTBS)

### Example 5

Synthesis of 4-tert.butyldimethylsilyloxy-5-(oct-1-ene)-cyclopent-2-ene-1-one (CTC-34)

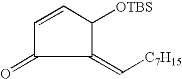

This compound was produced using the method described in Example 2 above, but with octanal substituted for the propionaldehyde used in the latter.

IR (KBr, Cm⁻¹): 2955, 2926, 2898, 1713, 1666

¹H NMR (CDCl₃, 400 MHz) δ: 7.39 (1H, d, J=6.0 Hz), 6.7 (1H, t, J=7.69), 6.40 (1H, d, J 6.0 Hz), 5.37 (1H, s), 2.3-2.5 (2H, m), 1.45-1.56 (2H, m), 1.2-1.4 (8H, m), 0.92 (9H, s), 0.87 (3H, t), 0.19 (3H, s), 0.16 (3H, s).

Mass (M/z): 323 (M⁺+1), 191 (M⁺−-OTBS)

### Example 6

Synthesis of butyldimethylsilyloxy-5-[but-1-ene]-cyclopent-2-ene-1-one (CTC-45)

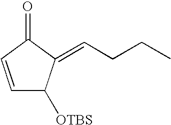

This compound was produced using the method described in Example 2 above but with butanal substituted for the propionaldehyde used in the latter.

IR (KBr, Cm⁻¹): 2958, 2931, 2858, 1711 and 1665

¹H NMR (CDCl₃, 400 MHz) δ: 7.39 (1H, m), 6.68 (1H, t, J=4.3 Hz), 6.40 (1H, d, J=5.88 Hz), 5.37 (1H, s), 2.25-2.46 (2H, m), 1.51-1.58 (2H, m), 0.99 (3H, t, J=7.36 Hz) 0.93 (9H, s), 0.19 (3H, s), 0.16 (3H, s)

### Example 7

(a) Preparation of 6-isopropylfulvene.¹⁸

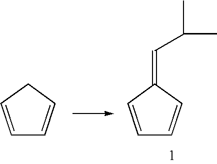

To a solution of freshly cracked cyclopentadiene (8.5 Cm³, 104 mmol) and isobutyraldehyde (3.8 cm³, 41.6 mmol) in reagent grade methanol (42 cm³) was added pyrrolidine (5.2 cm³, 62.4 mmol). Upon addition of the pyrrolidine the solution turned yellow and this mixture was stirred at room temperature for 2 min in an atmosphere of nitrogen. Once the reaction was complete glacial acetic acid 1(3.8 cm³) was added to the yellow solution. The reaction mixture was then diluted with ether (180 cm³) and water (180 cm³). The aqueous layer was further washed with ether (2×100 cm³), and the combined organic layers washed with water (20 cm³) and brine (20 cm³), dried (MgSO₄) and reduced in vacuo to yield 1 (5.0 g, 100%) as a yellow oil.

δ_{H}(300 MHz; CDCl₃; Me₄Si) 1.13 (6H, d, [symbol] 6.9, C(8)H, 2.95-3.07 (1H, m, C(7)H, 6.18-6.26 (1H, m, C(6)H), 6.44-6.55 (4H, m, ring protons); δ^{C}(75 MHz; CDCl₃; Me₄Si) 23.1 CH₃), 30.4, 119.3, 126.0, 130.8, 133.0, 149.8 (CH), 143.7(C).

(b) Preparation of 4-hydroxy-5-isobutylidene-cyclopent-2-enone.¹⁹

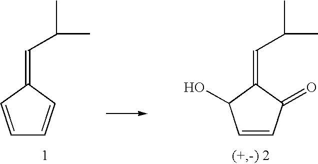

A solution of 1 (1.0 g, 8.30 mmol) and Rose Bengal (10 mg, 0.01 mmol) in reagent grade methanol (200 CM³) was stirred with a constant steady stream of oxygen bubbling through to saturate the solution. After 20 min the radiation (250W IR light bulb) was turned on with the oxygen flow continued. After 13 h the radiation was ceased and the methanol removed under reduced pressure. The crude product was purified by flash column chromatography (SiO₂; EtOAc: petroleum ether, 1:1) to yield (110 mg, 9%) of 2 as an orange oil and as a mixture of both geometric isomers. Vₘₐₓ(film)cm,⁻¹3399br (OH), 2963, 1701, 1657 (doubly conjugated 5-ring ketone²);δ_{H} (400 MHz; CDCl₃; Me₄Si) 1.04 and 1.11 (6 h, dd, J 10.6, 4.0 and 6.7, 9.0 (C(8)H), 1.84 (1H, br s, —OH), 2.93-2.99, 3.84-3.87 (1H, m, C(7)H), 5.07, 5.31 (1H, s, C(4)1), 6.17, 6.50 (1H, d, J 10.0 and 10.4, C(6)H, 6.35, 6.40 (1H, d, J 6.0 and 6.1, C(2)H), 7.38, 7.46 (1H, dd, J 2.5, 6.0 and 2.3, 6.0, C(3)H; δ_{C} (100 MHz, CDCl₃; Me₄Si) 22.1, 22.3, (CH₃), 25.9, 28.7, 70.4, 72.3, 136.5, 138.1, 145.7, 149.7, 156.8, 157.9 (CH), 134.3, 134.4, 195.2, 195.3 (C); m/z (EI) 152 M+, 29%), 109 (100).

(c) Preparation of tert-butyldimethylsilyl derivative (CTC-35)

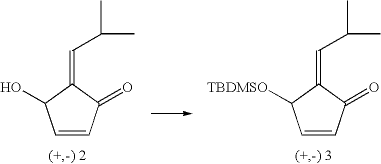

A solution of 2 (0.45 g, 2.96 mmol), in anhydrous dichoromethane (3.0 cm³) was added dropwise to a stirred solution of tert-butyldimethylsilylchloride (0.58 g, 3.80 mmol), triethylamiine (1.4 cm³, 10.36 mmol) and a catalytic amount of diethylaminopyridine (50 mg, 0.38 mmol) in anhydrous dichloromethane (3.0 cm³) at 0° C. The reaction mixture was then allowed to warm to room temperature and left stirring overnight in an atmosphere of nitrogen. After 70 h the reaction mixture was quenched with water (5 cm³ and the product extracted with dichloromethane (2×5 cm³). The combined organic layers were then dried (MgSO₄) and reduced in vacuo. The crude reaction mixture was then purified by flash column chromatography (SiO₂;EtOAc: petroleum ether, 1:10) to yield 3 (0.25 g, 32° h) as a yellow oil of one geometric isomer and (80 mg, 10%) of the other isomer also as a yellow oil. Vₘₐₓ(film)/cm⁻¹ 2959, 1712, 1663, 1111, 1072 (Si—O); 8H (400 CDCl₃;Me₄Si) 0.10 (6H, s, SiMe₂), 0.91, (9H, s, SiMe₃), 1.08 (6H, dd, J 6.6 and 8.5, C(8)H), 2.84-2.90 (1H, m, C(7)H), 5.36 (1H, s, C(4)H), 6.38 (1H, dd, J 6.1 and 1.1, C(2)H), 6.46 (1H, d, J 10.6, C(6)H, 7.37 (1H, dd, J 6.1 and 3.3, C(3)H); Sc (100 MHz; CDCl₃Me₄Si) −3.6, 22.0, 25.7, (CH₃), 28.3, 70.7, 136.2, 144.7, 157.9 (CH), 18.0, 134.4, 195.2 (C); m/z(EI) 266 (M⁺, 1%), 209 (100).

(Found: C, 67.70; H, 9.93. C₁₅H₂₆ O₂Si requires C, 67.62; H, 9.84%); Vₘₐₓ(film)/cm⁻¹ 2958, 1704, 1659, 1122, 1079 (Si—O); 8H (300 Mhz; CDCl₃; Me₄Si) 0.14 (6H s, SiMe₂), 0.93 (9H, s, SiMe₃), 1.01-1.06 (6H, m, C(8)H), 3.79-3.91 (1H, m, C(7)H), 5.13 (1H, s, C(4)H), 5.97 (1H, d, J 9.9, C(6)H), 6.31 (1H, d, J 6.0, C(2)H), 7.28 (1H, dd, J 2.4 and 6.0, C(3)H); δ_{C} (100 MHz, CDCl₃;Me₄Si) −4.1, 22.4, 25.7 (CH₃), 72.7, 137.6, 148.6, 157.1 (CH), 18.1, 134.8, 195.3 (C);m/z 266 (M⁺, 21%), 209 (90).

### Example 8

(a) Preparation of 5-(1-Methyl-octylidene)-cyclopenta-1,3-diene

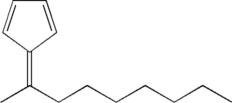

To a solution of 2-nonanone (4.5 cm³, 26.3 mmol) and cyclopentadiene (3.5 cm³, 42.6 mmol) in methanol (26.3 cm³) was added pyrrolidine (3.3 cm³, 39.45 mmol) and the mixture was stirred at room temperature under nitrogen. After 30 min acetic acid (2.4 cm³, 42.1 mmol) was added to the yellow solution and the mixture was diluted with ether and water (130 cm³ each). The aqueous portion was washed with ether (2×100 cm³) and the combined organics were washed with water and brine (25 cm³ each), then dried (MgSO₄) and concentrated in vacuo. The product was purified by flash column chromatography (SiO₂, 100% petroleum ether) to yield 3.14 g (63%) of the title compound. δ_{H} (400 MHz; CDCl₃; Me₄Si), 0.88 (3H, t, J 6.9, alkyl Me), 1.27-1.32 (8H, m, aliphatic chain), 1.52-1.59 (2H, m, CH₂ next to allylic CH), 2.20 (3H, s, allylic Me), 2.52 (2H, t, J 7.5, allylic CH), 6.45-6.52 (4H, m, ring protons). δ_{C} (100 MHz; CDCl₃; Me₄Si) 14.1 (CH₃), 20.9 (CH₃), 22.7 (CH), 29.2 (CH₂), 29.6 (CH), 29.7 (CH₂), 31.8 (CH), 36.9 (CH), 120.4 (CH), 120.7 (CH), 130.4 (CH), 130.7 (CH), 142.5(C), 154.5 (C).

(b) Preparation of 4-hydroxy-5-[1-methyl-oct-E/Z-ylidene]-cyclopent-2-enone, 2

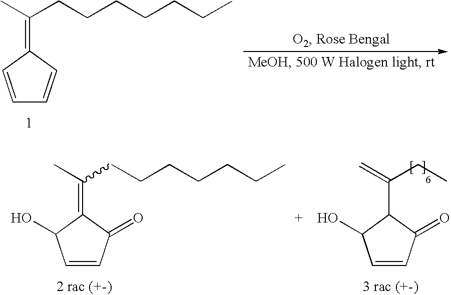

A solution of 1 (2.50 g, 13.1 mmol) and Rose Bengal (30 mg, 0.03 mmol) in reagent grade methanol (250 cm³) had a steady stream of oxygen bubbled through for 15 min to saturate the solution. The solution was irradiated with a 500W halogen light source for 12.5 h. The reaction mixture was filtered and the methanol removed under reduced pressure. The product was purified by flash column chromatography (SiO₂, EtOAc: petroleum ether, 3:7) to yield a mixture of 2 and 3 (0.87 g, 30%) as a brown oil and 3 alone (0.19 g, 6%) of a brown oil of the isomerised product. Mixed product; vₘₐₓ(film)/cm⁻¹ 3410 brs, 2926, 2856, 1698, 1635; g (400 MHz; CDCl₃; Me₄Si) 0.88 (3H, t, J 6.6, alkyl Me), 1.28-1.51 (10H, m, alkyl chain), 2.08-2.12 (2H, m, allylic CH₂ product 3), 2.29 (3H, s, allylic Me), 2.79 (2H, t, J 7.9, allylic CH, product 2), 3.30 (1H, d, J 6.4, C(5)H product 3), 4.97 (1H, brs, C(4)H product 3), 4.73 (1H, s) and 5.18 (1H, s)(protons at terminal alkene in 3), 6.28-6.35 (2H, m, C(2)H for both products), 7.31 (1H, dd, J 2.6 and 6.0, C(3)H product 2), 7.63 (1H, dd, J 2.4 and 5.7, C(3)H product ³);Sc (100 MHz; CDCl,; Me₄Si) Product 2; 133.2, 137.9, 155.0 (CH), 131.2, 156.5, 195.0 (C); Product 3; 14.1 (CH₃), 22.6, 27.2, 29.2, 31.8, 37.8, 114.6 (CH), 56.5, 71.3, 135.6, 163.3 (M, 145.3, 206.8 (C); m/z (EI) 222 (M⁺, 20%) and 151 (45); HRMS Found 222.16202, Calculated for C₁₄H₂₁O₂ 222.16199.

Single product: vₘₐₓ(film)/cm⁻¹ 3419 brs, 2927, 2856, 1704; 5 (400 MHz; CDCl₃; Me₄Si) 0.88 (3H, t, J 6.6, alkyl Me), 1.28-1.50 (10H, m, alkyl chain), 2.06-2.12 (2H, m, allylic CH), 3.30 (1H, d, J 6.5, C(5)H), 4.97 (1H, brs, C(4)H), 4.73 and 5.18 (2H, s, terminal alkene protons), 6.34 (1H, d, J 5.8, C(2)H), 7.63 (1H, dd, J 2.4 and 5.7, C(3)H); δ_{C} (100 MHz; CDCl₃; Me₄Si) 14.1 (CH₃), 22.6, 27.2, 29.2, 31.8, 37.8, 114.6 (CH₂), 56.5, 71.3, 135.6, 163.3 (CH), 145.3, 206.8 (C); m/z (EI) 222 (M⁺, 12%); HRMS Found 222.16158 Calculated for C₁₄H₂₂O₂ 222.16199.

(c) Preparation of tert-Butyl derivative of 2 and 3

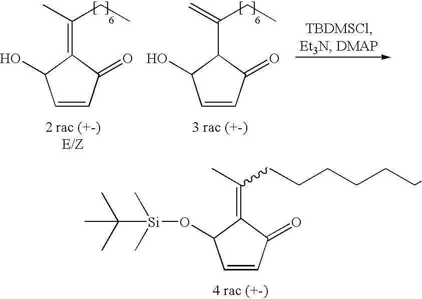

To a solution of the mixture of the alcohols (338 mg, 1.52 mmol) in anhydrous dichloromethane (2 cm³) was added a solution of tert-butyldimethylsilylchloride (0.3 g, 1.98 mmol), dimethylaminopyridine (0.02 g, 0.20 mmol) and triethylamine (0.75 cm³, 5.32 mmol) in dichloromethane (3 cm⁻¹) at 0° C. The solution was allowed to warm to room temperature over an atmosphere of nitrogen. After 50 h a further portion of tert-butyldimethylsilylchloride (0.11 g, 0.76 mmol) and triethylamie (0.3 cm³, 2.1 mmol) in dichloromethane (1 cm³) was added dropwise to the reaction mixture at 0° C. and allowed to warm to room temperature. After 94 h the reaction was quenched with water (5 cm³), and the product extracted with dichloromethane (3×5 cm³), dried (MgSO₄) and evaporated under reduced pressure. The product was purified by flash column chromatography (SiO₂; 0.6:10 EtOAc: petroleum ether then 0.75:10 EtOAc: petroleum ether) to yield 4 (37.6 mg, 7.3%) as a yellow oil of one geometric isomer. vₘₐₓ(film)/cm⁻¹ 2929, 2857, 1698, 1643; δ_{H} (400 M; CDCl₃; Me₄Si) 0.06 (3H, s, SiMe), 0.11 (3H, s, SiMe), 0.89 (9H, s, 'BuSi), 1.24-1.48 (13H, m, aliphatic H's), 2.00 (3H, s, allylic Me), 2.75-2.80 (2H, m, allylic CH), 5.29 (1H, s, C(4)H), 6.26 (1H, d, J 6.0, C(2)H), 7.23 (1H, dd, J 2.5 and 6.0, C(3)H; δ_{C} (100 MHz; CDCl₃; Me₄Si) −3.4, −2.8, 14.4, 22.1, 26.1 (CH₃), 23.0, 28.6, 29.5, 30.1, 32.2, 33.6 (CH), 73.1, 137.9, 155.2, (CH, 18.4, 131.9, 155.5, 195.4 (C); m/z (ED 336 (M⁺, 29%), 279 (87, loss of 'Bu group)

Activity of Compounds in Accordance with the Invention

Preferred compounds of the present invention have activity in one or more of the assays described in Examples 9 to 14 below. Desirably, this activity is greater than that of cyclopent-2-en-1-one (which can be assayed for comparison). Certain compounds of the present invention may be advantageous in avoiding the affect of lowering blood pressure that is associated with various prostaglandins. An assay for this is set out in Example 15 below.

### Example 9

Effect of Inventive Compounds on the Reactivity of Transcription Factors HSF and NF-κB

Methods: Human lymphoblastoid Jurkat T cells were grown at 37° C. in a 5% CO₂ atmosphere in RPM1 1640 medium (GIBCO BRL, Gaithersburg, Md.) supplemented with 10% fetal calf serum (FCS, Hyclone Europe Ltd, UK) 2 mM glutamine and antibiotics according to the method described by A. Rossi et al. (Proc. Natl. Acad. Sci. USA 94: 746-750, 1997). The test compounds were stored as a 100% ethanolic stock solution (100 mM) or in DMSO (100 mM and diluted to the appropriate concentration in culture medium at the time of use. Cells were treated with different concentrations of test compound for 1 hour and then stimulated with 12-O-tetradecanoylphorbol-13-acetate (TPA, 25 ng/ml), which is a strong inducer of NF-κB. Control cells received an equal amount of control diluent. After 3 hours whole-cell extracts were prepared and subjected to analysis of DNA-binding activity by EMSA (Electrophoretic Mobility Shift Assay) for detection of HSF or NF-κB activation, according to the method described by A. Rossi et al. (Proc. Natl. Acad. Sci. USA 94: 746-750, 1997).

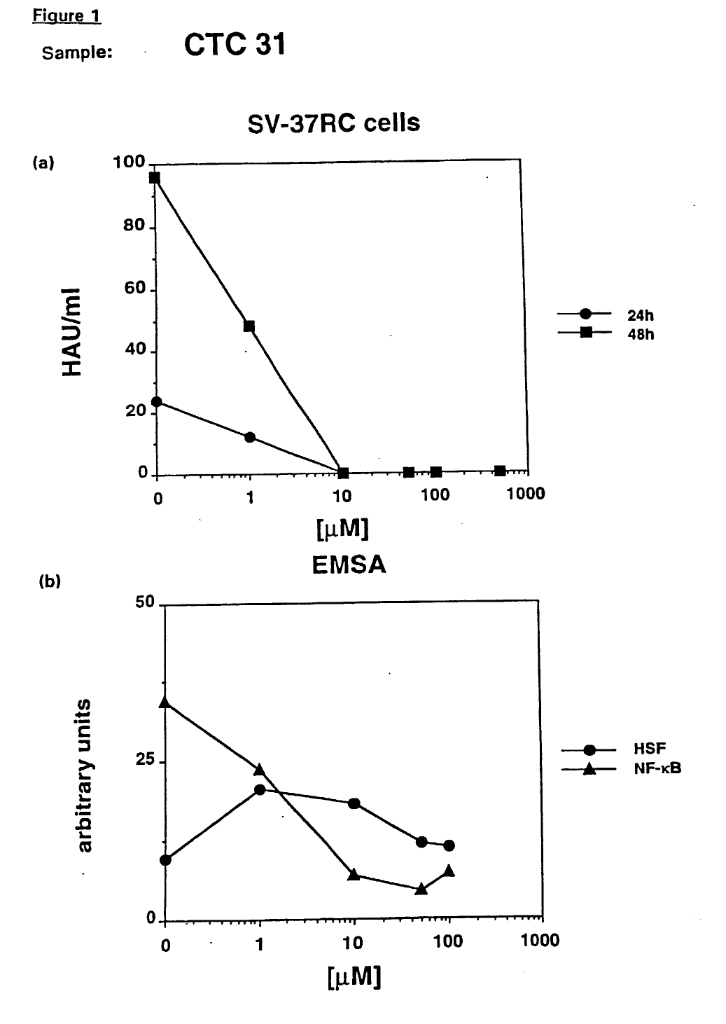

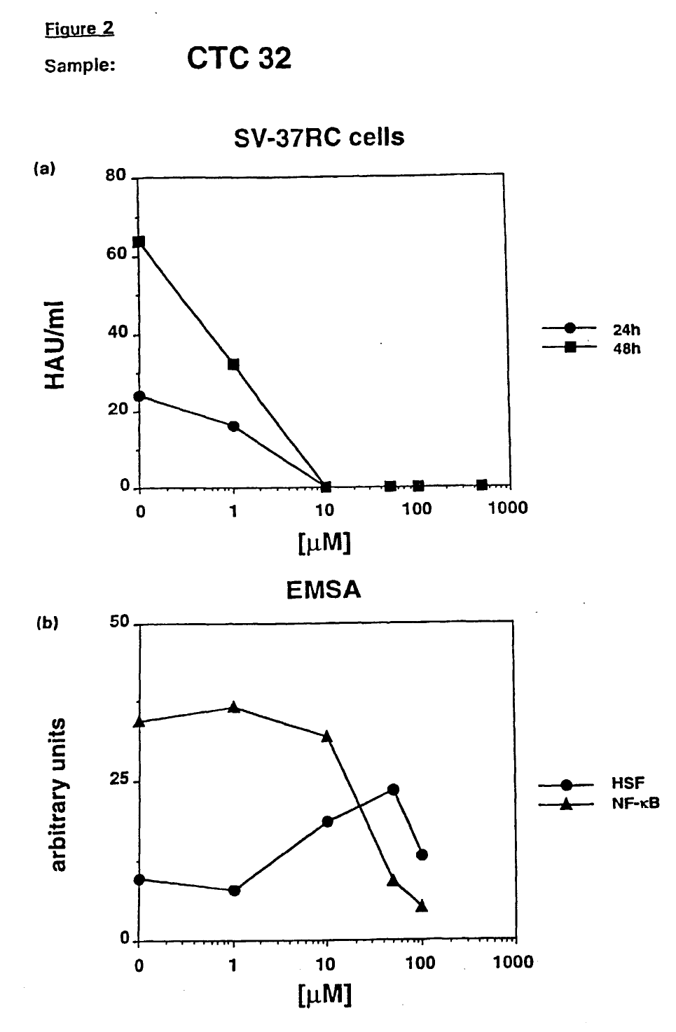

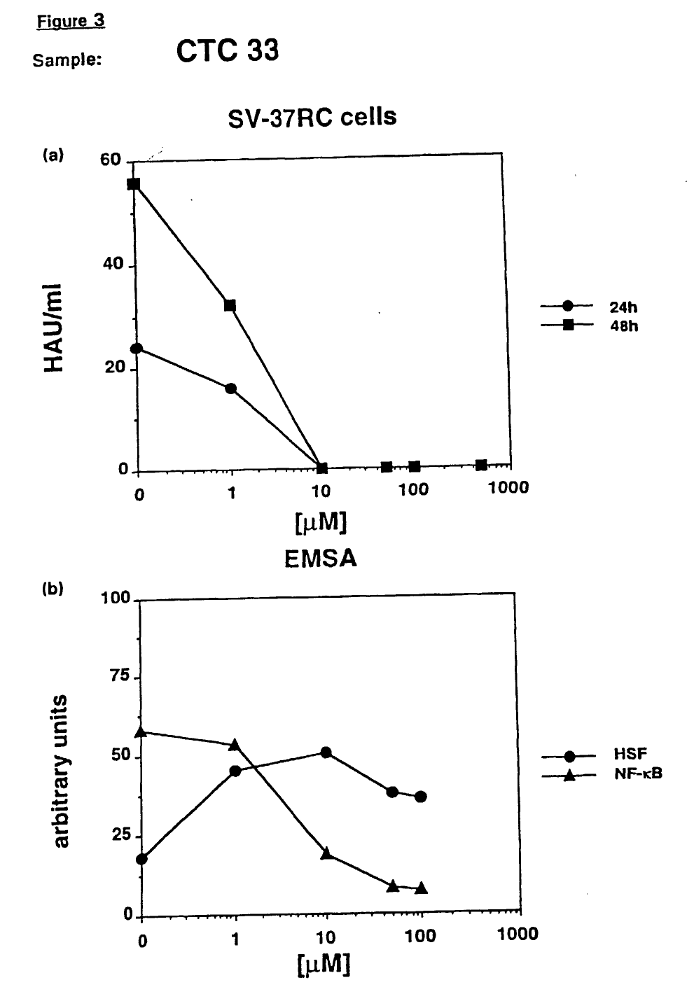

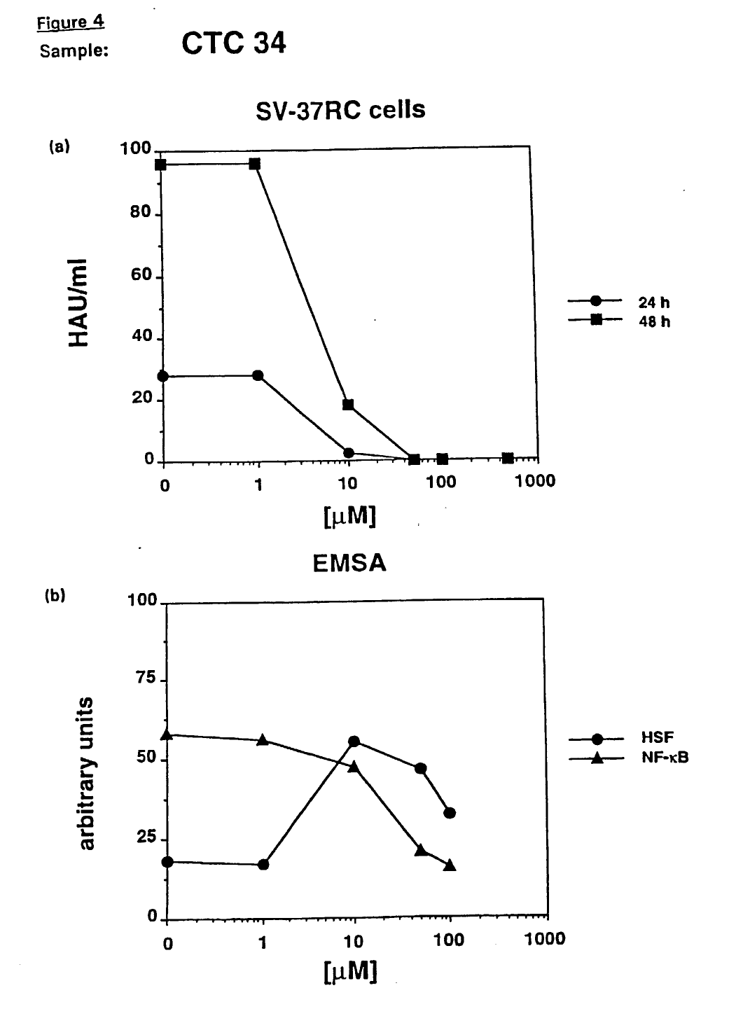

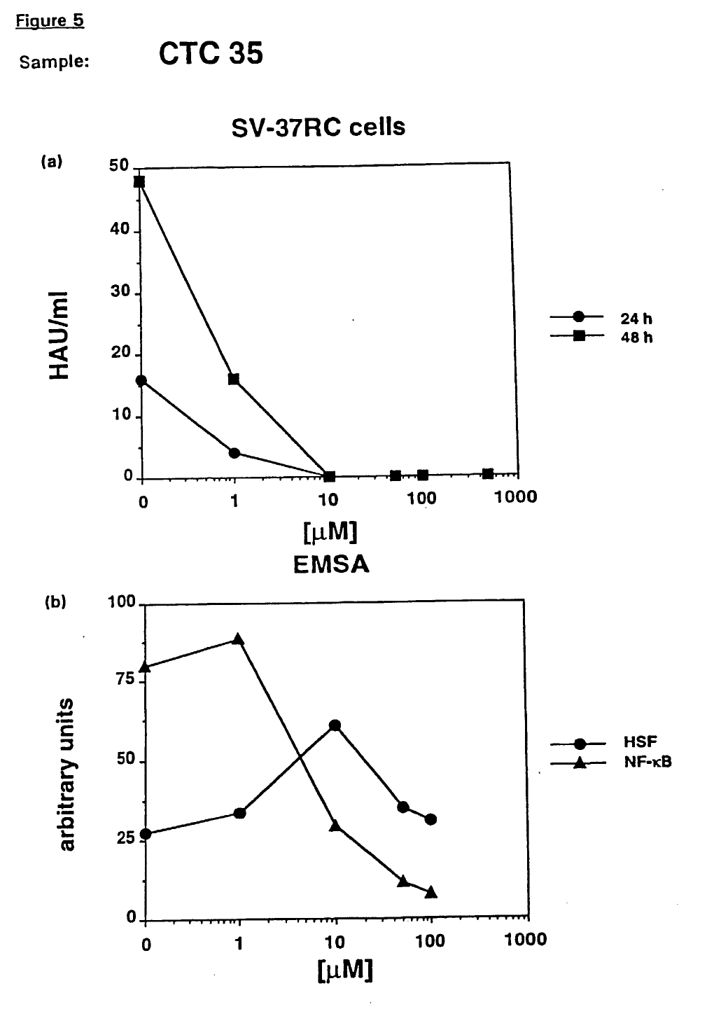

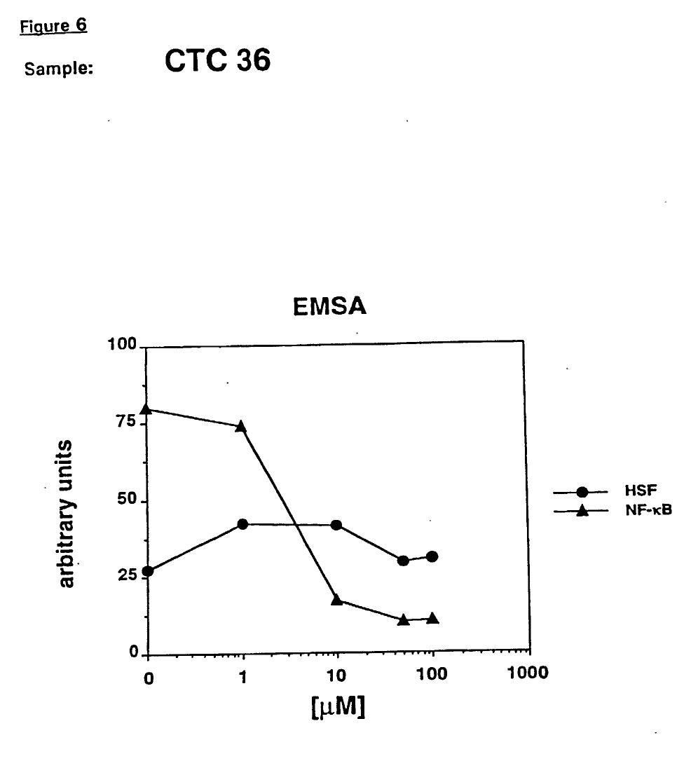

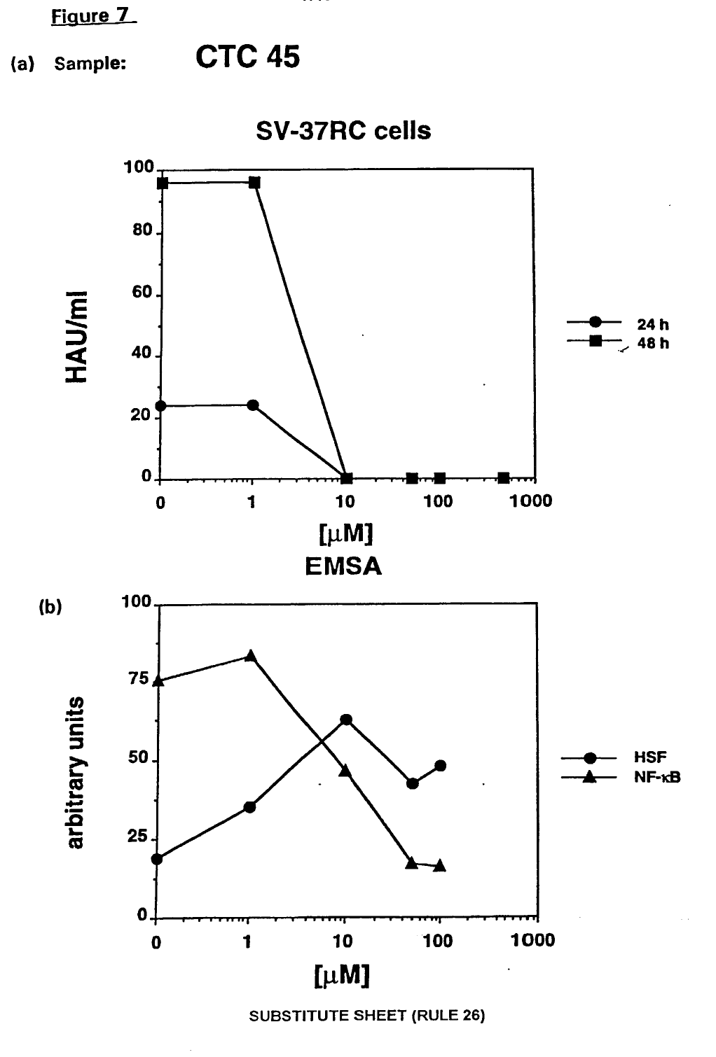

Specificity of protein-DNA complexes was verified by immunoreactivity with polyclonal antibodies specific for p65 (Rel A) or for HSF-1, for NF-κB and HSF respectively. Quantitative evaluation of NF-κB- and HSF-DNA complex formation was determined by Molecular Dynamics Phosphorlmager (MDP) analysis and was expressed in arbitrary units, as described in A. Rossi et al.(J. Biol. Chem. 273: 16446-16452, 1998). The results from representative experiments are shown in FIGS. 1(b), 2(b), 3(b), 4(b), 5(b), 6 and 7(b) for compounds CTC-31, CTC-32, CTC-33, CTC-34, CTC-35, CTC-36 and CTC-45, as identified above. These results show that all of these latter compounds are potent inhibitors of NF-κB.

### Example 10

Effect of CTC-35 on the Replication of Herpes simplex Virus Type 1

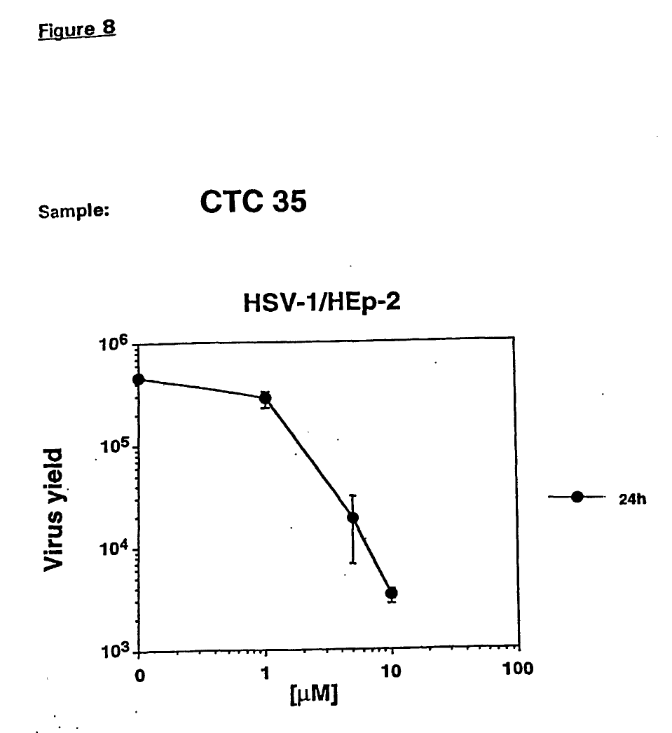

Methods: Human Hep-2 laryngeal carcinoma cells and monkey VERO cells were grown at 37° C. under the conditions described in Example 9 for T cells. Cell viability was determined by dye exclusion technique or by 3-(4,5-dimethylthiazol-2-yl)-2,5-diphenyl-tetrazolium bromide (MTT, Sigma Chemical Co.) to MTT formazan assay, as described by F. Denizot and R. Lang (J. Immunol. Methods 89: 271-277, 1986). Herpes simplex virus type one (HSV-1), strain F grown in VERO cells, was used at a multiplicity of infection of 10 plaque forming units (PFU) per cell. Confluent Hep-2 cell monolayers were infected with HSV-1 for 1 h at 37° C. After this time, virus inocula were removed and cells incubated in RPMI 1640 medium containing 2% FCS. Different concentrations of CTC-35 were added to the culture after the 1 h adsorption period, and maintained in the medium for the duration of the experiment. Control medium contained the same concentration of ethanol diluent, which did not affect cell metabolism or virus replication. HSV-1 virus titres were determined 24 hours after infection by cytopathic effect 50% (CPE 50%) assay on confluent VERO cells monolayers in 96-well tissue culture plates (six dilutions for each sample, eight wells for each dilution), as described by F. Pica et al. (Antiviral Res. 20: 193-208, 1993). The dilution that gives 50% cytopathic effect were determined by the interpolating procedure of Reed and Muensch, as described by E. Rodriguez-Boulan Methods Enzymol. 98: 486-501, 1983). The results from a representative experiment are shown in FIG. 8 and indicate that CTC-35 is a potent inhibitor of HSV-1 virus replication, with an ID₅₀=1.51 μm (ID50=50% inhibitory dose/concentration).

### Example 11

Effect of Inventive Compounds on the Replication of Sendai Virus

Methods: Monkey kidney 37RC cells were grown at 37° C. under the conditions described in Example 10 for T cells. The parainfluenza Sendai virus (SV) was grown in the allantoic cavity of 10-day-old embroynated eggs. Viral titre was expressed in haemagglutinating units (HAU) per ml; haemagglutinin titration was done according to standard procedures using human 0 Rh+erythrocytes, as described in C. Amici et al. (J. Virol. 68: 6890-6899, 1994). Confluent monolayers of 37RC cells were infected with SV virus (5 HAU/10⁵ cells) for 1 h at 37° C., and then treated with different concentrations of test compounds. Virus yield at 24 hours after infection was determined in the supernatant of infected cells by HAU titration. The results of representative experiments are shown in FIGS. 1(a), 2(a), 3(a), 4(a), 5(a), 7(a) for compounds CTC-31, CTC-32, CTC-33, CTC-34, CTC-35, and CTC-45, as identified above. These results show that all of these latter compounds are potent inhibitors of Sendai virus replication.

The ID₅₀ (the 50% inhibitory dose/concentration) values at 24 hours and the TD₁₀₀ (the dose or concentration at which the tested compound was 100% toxic to uninfected cells, determined visually by microscopy) for the tested compounds is given below and shows that the anti viral effect of the tested compounds took place at a concentration at which they were non-toxic to the 37RC cells.

| Compound | ID₅₀/μM | TD₁₀₀/μM |
|---|---|---|
| CTC-31 | 1 | 50 |
| CTC-32 | 2 | 10 |
| CTC-33 | 1.5 | 50 |
| CTC-34 | 3 | 50 |
| CTC-35 | 0.4 | 50 |
| CTC-45 | 3 | 50 |

### Example 12

Effect of CTC-35 on Infection with Influenza Virus.

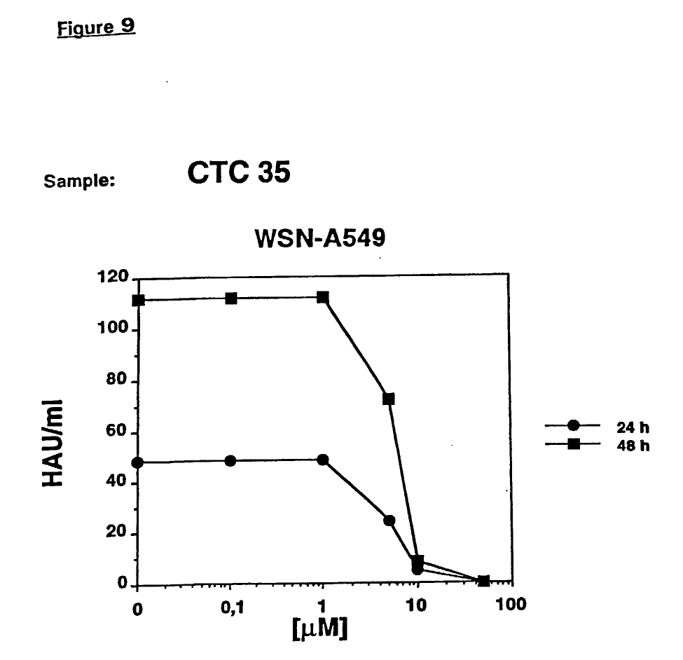

Human lung adenocarcinoma A549 cells were grown at 37° C. in RPMI-1640 medium, supplemented with 10% fetal calf serum TICS, Gibco) and antibiotics. Influenza A virus A/WSN/33 (H₁N₁) (WSN virus) was grown in the allantoic cavity of 10-day-old embryonated eggs. Virus titers were determined by hemagglutinin titration, according to standard procedures (Pica F, Palamara A T, Rossi A, De Marco A, Amici C and Santoro M G: Δ¹²-Prostaglandin J₂ is a potent inhibitor of influenza A virus replication. Antimicrob. Agents Chemther., 44: 200-204, 2000). Confluent A549 monolayers were infected with WSN virus (10 HAU/10⁵ cells) for 1 h at 37° C. After this time, viral inoculum was removed and cells were treated with different concentrations of CTC35 or ethanol-diluent. Viral yields were determined 24 and 48 h post infection (p.i.) and expressed as HAU/ml (each point represents the mean of duplicate samples). The results of these experiments are given in FIG. 9 and show CTC-35 to be a potent inhibitor of influenza virus infection, with an ID₅₀ (the 50% inhibitory dose/concentration) of 511M at 24 hours exposure.

### Example 13

MTT Assay

Cell viability was determined by the 3-(4,5-dimethylthiazol-2-yl)-2,5-diphenyltetrazolium bromide MIT) assay. Uninfected A549 (7.5×10⁴ cells/well in 96 well plates) or 37RC cells (2.5×10⁴ cells/well in 96 well plates) were treated with different concentrations of CTC35 or ethanol diluent for 24 hours. After this time, 10 ml of a 0.5% MIT solution in PBS was added to the monolayers and the mixture was incubated for 2 h at 37° C. Reduced MTT (formazan) was extracted from cells by adding 100 μl of acidic isopropanol containing 10% Triton X-100, and formazan absorbance was measured in an ELISA microplate reader at two different wavelengths (540 and 690 nm).

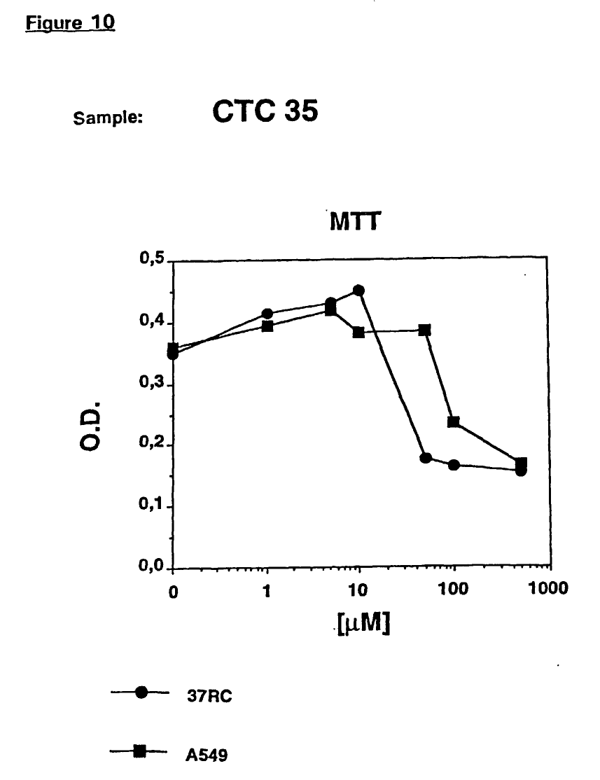

The results from quadruplicate samples are shown in FIG. 10 and indicate that the anti viral activity shown in examples 11 and 12 occurs at CTC-35 concentrations that are non-toxic to the cells, as the LD₅₀ (Lethal dose/concentration 50%) was determined to be 90 μM for A549 cells and 30 μM for 37RC cells and, thus, significantly higher than the ID₅₀ (the 50% inhibitory dose/concentration) values determined for both compounds.

### Example 14

Assaying for Anti-Inflammatory Effect

Immune cells such as neutrophils and macrophages are activated in response to injury and infection. When activated they produce nitric oxide and superoxide radicals to kill foreign cells and cancer cells. They also produce a variety of cytokines and chemokines to cause further recruitment of immune cells in a cascade leading to the cardinal symptoms of inflammation; heat, redness, swelling, pain, and loss of function.

A key signalling step in the activation of the immune cells is the transcription factor nuclear factor κB (NF-κB) (16). NF-κB regulates the transcription of a spectrum of pro-inflammatory genes such as IL-1, IL-2, TNF-α, ICAM-1, VCAM-1, and E-selectin as well as the inducible form of nitric oxide synthase (iNOS) and cyclo-oxygenase II.

Thus the activation of NF-κB occupies a critical position in the inflammatory cascade. A test compound can be tested for its effects on the induction of iNOS in a mouse macrophage model.

Mouse macrophages of the cell line RA W264.7 can be stimulated with gamma interferon and 0.1 U/ml of bacterial lipopolysaccharide (LPS) in 96-well plates (17). The induction of iNOS can be measured by determination of the levels of nitrite (NO₂⁻) formed in the supernatant, using the Griess reagent.

It can be determined whether or not Compound X has an inhibitory effect on nitrite formation (preferably at submicromolar concentrations). The natural cyclopentenone prostaglandin PG-J₂ can be used for comparison. (IC50 values obtained for PGJ₂ and a test Compound can be compared).

If the results of the experiment indicated that the induction of the pro-inflammatory iNOS genes by interferon gamma and LPS treatment is suppressed by Compound X, the most likely explanation is that the test compound is inhibiting the activation of the NF-κB pathway.

### Example 15

Assaying to Determine Whether or not Compound X Lowers Blood Pressure

Most prostaglandins have strong effects on vascular smooth muscle, and will lower blood pressure in animals and humans. A compound can be tested for its effect on the blood pressure of the anaestheized rat. Prostaglandins A₁ and E₁ can be used for comparison.

Male Wistar rats were anaesthetized and test drugs can be infused intravenously. Blood pressure and heart rate can be recorded from the femoral artery. Prostaglandins A₁ and E₁ cause dose-dependant falls in blood pressure in doses from 30 μg/kg/min. It can be determined whether or not a test compound affects blood pressure at various dosages. As a control, solvent alone can be used.

If a compound does not cause significant changes in blood pressure, it may be devoid of the generalised effects on smooth muscle characteristic of natural cyclopentenone prostaglandins.

### General Remarks

The foregoing description of the invention is merely illustrative thereof and it should therefore be appreciated that various variations and modifications can be made without departing from the spirit or scope of the invention as set forth in the accompanying claims.

Where preferred or optional features are described in connection with particular aspects of the present invention, they shall be deemed to apply mutatis mutandis to other aspects of the invention unless the context indicates otherwise.

All documents cited herein are hereby incorporated by reference, as are any citations referred to in said documents.

### REFERENCES

1. Feige U, Morimoto R, Yahara I, Polia B S. Stress-inducible Cellular Reponses. Birkhaüser Verlag, Basel Boston Berlin, 1996.

2. Marber M S, Walker J M, Latchman D S, 'Yellon D M. J. Clin. Invest. 7 93, 1087-1094, 1994.

3. Feinstein D L et al. J. Biol. Cha 271, 17724-17732, 1996.

4. Amici C, Giorgi C, Rossi A, Santoro M G. J. Virol 68, 6890-6897, 1994.

5. Santoro M G, in Stress-inducible Cellular Responses. (Fiege U et al. eds, Birkhaüser Verlag, Basel Boston Berlin) pp. 337-357, 1996.

6. Santoro MG, Garaci 9, Amici C. P.N.A.S. USA 86, 8407-8411, 1989.

7. Amici C, Sistonen L, Santoro M G, Morimoto R I. P.N.A.S. USA 89, 6227-6231, 1992.

8. Santoro M G, Benedetto A. Carruba G, Garaci E, Jaffe B. Science 209, 1032-1034, 1980.

9. Santoro M G, Trends Microbiol. 5, 276-281, 1997.

10. Rozera C, Carattoli A, De Marco A, Amici C, Giorgi C, Santoro M G J. Clin. Invest 97; 1795-1803, 1996.

11. Rossi A, Elia G, Santoro M G. P.N.A.S. USA 94, 746-750, 1997.

12. Thanos D, Maniatis T. Cell 80, 529-532, 1995.

13. Rossi A, Elia G, Santoro M G. J. Biol. Chem. 271, 32192-32196, 1996.

14. Shield M J. Pharmacol. Ther. 65, 125-137, 1995.

15. Sinclair S B et al. J. Clin Invest. 84, 1063-1067, 1989.

16. Baeuerle P A and Henkel T (1994). Function and Activation of NF-Kappa B in the Immune System. Annual Reviews of Immunology 12: 141-179.

17. Colville-Nash PR et al. (1998). Inhibition of Inducible Nitric Oxide Synthase by Peroxisome Proliferator-Activated Receptor Agonists: Correlation with Induction of Heme Oxygenase 1. Journal of Immunology 161, 978-984

18. K. J. Stone, R. D. Little, JOC, 1984, 49, 1849-1853.

19. A. Kawamoto, H. Kosugi, H. Uda, Chem. Lett., 1972, 807-810.

1. A compound having the formula I:— 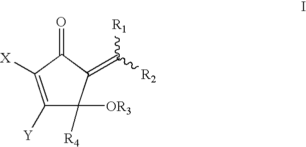
wherein:—
R₁ is H, or a substituted or unsubstituted alkyl or alkenyl group containing 1 to 3 carbon atoms;
R₂ is a substituted or unsubstituted alkyl, alkenyl, alkynyl, aryl, aralkyl aralkenyl, or aralkynyl group, optionally including at least one heteroatom in its carbon skeleton, and containing 1-12 carbon atoms;
R₃ is a substituted or unsubstituted alkyl, alkenyl, alkynyl, aryl, aralkyl aralkenyl, or aralkynyl group, optionally including at least one heteroatom in its carbon skeleton, and containing 1-12 carbon atoms, or a silyl group;
R is H, or an alkyl group containing 1-3 carbon atoms;
X and Y, independently, are H, a halogen, or an alkyl group containing 1-3 carbon atoms; and
R₂ can be cis- or trans- with respect to the carbonyl carbon in the cyclopentene ring.

2. A compound as claimed in claim 1, wherein R₂ and/or R₃ is substituted with at least one ═O, —OR —COOR₅, and/or halogen atom or group, wherein R is hydrogen or an alkyl group containing up to 4 carbon atoms.

3. A compound as claimed in claim 2, wherein R₅ is hydrogen or a methyl group.

4. A compound as claimed in any of the preceding claims, wherein R₂ and/or R₃ includes an oxygen, nitrogen or sulphur atom in its carbon skeleton.

5. A compound as claimed in any one of the preceding claims, wherein R₁ is hydrogen or an unsubsituted alkyl group.

6. A compound as claimed in claim 5, wherein R₁ is hydrogen or a methyl group.

7. A compound as claimed in any of the preceding claims, wherein R₄ is hydrogen or a methyl group.

8. A compound as claimed in any of the preceding claims, wherein R₂ and/or R₃ is a substituted or unsubstituted, straight chain, branched and/or cyclo-alkyl, alkenyl, or alkynyl group.

9. A compound as claimed in any one of claims 1-7, wherein R₂ is a substituted or unsubstituted heterocylic, aralkyl or aryl group.

10. A compound as claimed in claim 9, wherein R₂ is a substituted or unsubstituted phenyl, thiophene or pyridinyl group.

11. A compound as claimed in claim 10, wherein R₂ is an unsubstituted thiophene, pyridinyl, phenyl, dimethylphenyl, halophenyl or alkoxyphenyl group.

12. A compound as claimed in claim 11, wherein R₂ is a fluorophenyl group or a methyloxyphenyl group.

13. A compound as claimed in any one of claims 1-8, wherein R₂ is a substituted or unsubstituted alkyl or alkenyl group including up to 10, 9, 8, 7, 6, 5, 4, or 3 carbon atoms.

14. A compound as claimed in claim 13, wherein R₂ contains 7, 3 or 2 carbon atoms and is an alkyl group.

15. A compound as claimed in claim 14, wherein R₂ is C₇H₁₅,iso-propyl, or ethyl.

16. A compound as claimed in any of the preceding claims, wherein R₃ is a substituted or unsubstituted alkyl or aralkyl group.

17. A compound as claimed in any of the preceding claims, wherein R₃ includes a carboxyl and/or a carbonyl group.

18. A compound as claimed in any of the preceding claims, wherein R₃ is a substituted or unsubstituted straight chain, branched and/or cyclo-alkyl group and contains 5, 6, 7 or 8 carbon atoms when it includes or is a cyclo-alkyl group and 5 or fewer carbon atoms when it is a straight chain or branched alkyl group.

19. A compound as claimed in claim 16, wherein R₃ is a substituted or unsubstituted aralkyl group containing 6, 7 or 8 carbon atoms.

20. A compound as claimed in any of the preceding claims, wherein R₃ includes a cyclohexyl or phenyl group.

21. A compound as claimed in any of claims 1-17, wherein R₃ is a succinyl group or a derivative.

22. A compound as claimed in claim 22, wherein R₃ is a 2-methysuccinyl, a 2-carboxyphenycarbonyl, or a 2-carboxycyclohexylcarbonyl group.

23. A compound as claimed in any one of claims 1-15, wherein R₃ is a tri(organo)silyl group.

24. A compound as claimed in claim 23, wherein each of the organo-groups is a substituted or unsubstituted alkyl, aryl and/or aralkyl group, optionally including at least one heteroatom in its carbon skeleton.

25. A compound as claimed in claim 24, wherein each alkyl group contains from 1 to 5 carbon atoms.

26. A compound as claimed in claim 24, wherein each aryl or aralkyl group contains at least 6 or 7 carbon atoms respectively.

27. A compound as claimed in claim 26, wherein each aryl group is phenyl and each aralkyl group is benzyl.

28. A compound as claimed in any of claims 23-27, wherein at least one of said organo-groups is substituted with one or more ═O, —OR₅, —COOR₅ and/or halogen (preferably fluorine) group or atom, wherein R₅ is hydrogen or an alkyl group containing up to 4 carbon atoms.

29. A compound as claimed in any of the preceding claims, wherein R₅ is a tert.butyldimethylsilyl group.

30. A compound as claimed in any of the preceding-claims having activity in respect of one or more of the following:
a) activating HSF
b) inhibiting NF-κB
c) inhibiting the replication of HSV-1
d) inhibiting the replication of Sendai virus
e) inhibiting influenza virus.

31. A compound as claimed in claim 30, having greater activity than cyclopent-2-ene-1-one in one or more of a), b), c), d) or e).

32. A compound as claimed in any of the preceding claims for use in medicine.

33. A compound as claimed in claim 32, for treating a viral-mediated disorder.

34. A compound as claimed in claim 32, for treating a bacterial-mediated disorder.

35. A compound as claimed in claim 32, for treating a disorder mediated by radiation.

36. A compound as claimed in claim 32, for treating an inflammatory disorder.

37. A compound as claimed in claim 32, for treating a disorder of the immune system.

38. A compound as claimed in claim 32, for treating ischemia.

39. A compound as claimed in claim 32, for treating arteriosclerosis.

40. A compound as claimed in claim 32, for treating a disorder involving cell proliferation.

41. A compound as claimed in claim 40, wherein the disorder is a cancer.

42. A compound as claimed in claim 32, for treating a disorder involving damage to cells or killing of cells.

43. A compound as claimed in claim 32, for treating diabetes.

44. A compound as claimed in claim 32, for treating a disorder affecting an aquatic organism.

45. A compound as claimed in claim 32, for treating oxidative stress, use as an anti-oxidant, use in combating the effects of ageing, or treating a degenerative disease.

46. A compound as claimed in claim 45, wherein the degenerative disease is neuro-degenerative, preferably BSE, new variant CJD, or Alzheimer's disease.

47. A compound as claimed in claim 32, for treating burns, a disorder involving calcium loss or deficiency, or use in promoting wound healing.

48. The use of a compound according to any of claims 1 to 31 as a research tool for the analysis of one or more of the following: HSF, NF-κB, the heat shock response, viral replication, viral-mediated disorders, bacterial-mediated disorders, disorders mediated by radiation (e.g. by UV-radiation), inflammatory disorders, disorders of the immune system, ischemia, arteriosclerosis, disorders involving cell proliferation, disorders involving damage to, or killing of cells, or diabetes.

49. A pharmaceutical composition comprising a compound according to any of claims 1 to 47 and optionally including a pharmaceutically acceptable carrier.

50. A composition as claimed in claim 49 for use in medicine, preferably for treating a disorder as recited in any one of claims 33-47.

51. A food for an aquatic organism comprising a compound according to any of claims 1 to 31.

52. An aquatic environment (e.g. an aquaculture) comprising a compound according to any of claims 1 to 31.

53. Use of a compound as claimed in any of claims 1-31, for the preparation of a medicament for treating a viral-mediated disorder, a bacterial-mediated disorder, a disorder mediated by radiation, an inflammatory disorder, a disorder of the immune system, ischemia, arteriosclerosis, a disorder involving cell proliferation, cancer, a disorder involving damage to cells or killing of cells, diabetes, oxidative stress, a degenerative disease, burns, a disorder involving calcium loss or deficiency, or a disorder effecting an aquatic organism.

54. Use of a compound as claimed in any of claims 1-31 for the preparation of a medicament for use as an anti-oxidant, in promoting wound healing or use in combating the effects of ageing.

55. Use of a compound as claimed in any of claims 1-31 for the preparation of a medicament for use in treating a neuro-degenerative disease, preferably BSE, new variant CJD, or Alzheimer's disease.

56. A method of treating a viral-mediated disorder, a bacterial-mediated disorder, a disorder mediated by radiation, an inflammatory disorder, a disorder of the immune system, ischemia, arteriosclerosis, a disorder involving cell proliferation, cancer, a disorder involving damage to cells or killing of cells, diabetes, oxidative stress, a degenerative disease, the effects of ageing, burns, a disorder involving calcium loss or deficiency, or a disorder effecting an aquatic organism, comprising administering a compound as claimed in any one of claims 1-31 or a composition as claimed in claim 49 to a subject suffering from one or more of said conditions, in an amount effective to at least ameliorate at least one of said conditions.

57. A method of promoting wound healing, comprising administering a compound as claimed in any one of claims 1-31 or a composition as claimed in claim 49 to a wounded subject in an amount effective to promote wound healing.

58. A method as claimed in claim 56, wherein the degenerative disease is a neuro-degenerative disease, preferably BSE, new variant CJD, or Alzheimer's disease.

59. A method for preparing a compound as claimed in any of claims 1-47 comprising reacting a compound of formula II:— 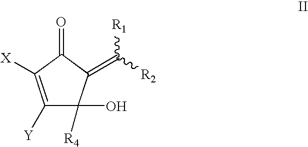
with a silyl chloride, succinic anhydride, or a derivative of succinic anhydride, preferably in the presence of a base and, more preferably, also in the presence of an alkyl amino pyridine, wherein R₁ and R₂ are as defined in any one of claims 1-44, the silyl group in the silyl chloride is, preferably, as defined in any one of claims 23-29 and the succinic anhydride derivative is selected to provide the required group R₃.

60. A method of preparing a compound of formula III:— 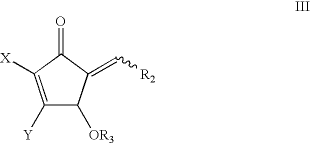
including the steps of:—
(a) reacting a compound of formula IV 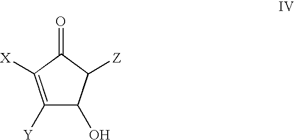
with a silyl chloride, succinic anhydride or a succinic anhydride derivative, preferably in the presence of a base and, more preferably, also in the presence of an alkyl amino pyridine, to prepare a compound of formula V:— 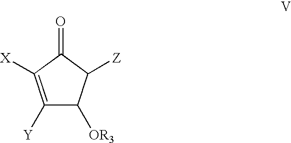
and
(b) reacting a compound of formula VI:— 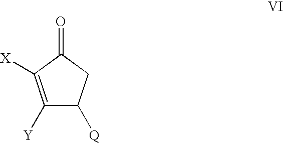
with R₂CHO in the presence of a base to give a compound of formula VII:— 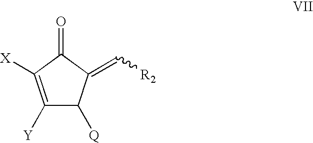
wherein;
when step (a) is carried out before step (b) Z is hydrogen, Q is OR, and the bonds between Z, Q and the cyclopentene ring are single bonds;
when step (b) is carried out before step (a), Q is an oxygen atom or a hydroxyl group, Z is CR₂ and the carbon atom in the CR₂ group is bonded to the cyclopentene ring by a double bond;
when Q is oxygen, the bond between the oxygen atom and the cyclopentene ring is a double bond and it is reduced to a hydroxyl group before step (a) is carried out;
X, Y, R₂, R₃ and the silyl group in the silyl chloride are as defined in any of claims 1-29 above; and
the succinic anhydride derivative is selected to provide the required group R₃.

61. A compound prepared or preparable by a method as claimed in claim 59 or 60, preferably for use in medicine.

62. A composition comprising a compound as claimed in claim 61 and a pharmaceutically acceptable carrier.

63. A compound as claimed in any of claims 147, having the formula of CTC-35 given in the specification.

64. A composition as claimed in any of claims 49, 50, 51 and 60, wherein the compound has the formula given for CTC-35 in the specification.

65. A plant treatment composition comprising a compound as claimed in any one of claims 1-31, 61 and 63 and a suitable carrier.

66. A composition as claimed in claim 65, wherein the carrier is selected so that the composition can be applied by spraying.

67. A method of treating a plant, comprising contacting the plant with a compound as claimed in any one of claims 1-31, 61 and 63, or a composition as claimed in claim 65 or 66, to treat or prevent a disorder in the plant.

68. A method as claimed in claim 67, wherein the disorder is a viral disorder.
